(19) United States
(12) Patent Application Publication
(10) Pub. No.: US 2010/0331858 A1
(43) Pub. Date: Dec. 30, 2010

(54) SYSTEMS, DEVICES, AND METHODS FOR ROBOT-ASSISTED MICRO-SURGICAL STENTING

(75) Inventors: Nabil Simaan, Nashville, TN (US); Howard Fine, Long Branch, NJ (US); Wei Wei, New York, NY (US); Stanley Chang, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/811,506

(22) PCT Filed: Jan. 30, 2009
(86) PCT No.: PCT/US09/32657

§ 371 Date: Sep. 1, 2010

Related U.S. Application Data
(63) Continuation-in-part of application No. 61/024,835, filed on Jan. 30, 2008.
Application No. 61/024,835 is a continuation-in-part of application No. 61/042,198, filed on Apr. 3, 2008.
Application No. 61/042,198 is a continuation-in-part of application No. 61/046,178, filed on Apr. 18, 2008.

(51) Int. Cl. A61B 19/00; A61F 2/84

(52) U.S. Cl. 606/130; 623/1.11

(57) ABSTRACT

Systems, devices, and methods for robot-assisted microsurgical stenting are described herein. In some embodiments a tele-robotic microsurgical system for eye surgery include: a tele-robotic master and a slave hybrid-robot; wherein the tele-robotic master has at least one master slave interface controlled by a medical professional; wherein the slave hybrid-robot has at least one robotic arm attached to a frame releasably attached to a patient's head; wherein the at least one robotic arm has a parallel robot and a serial robot; and wherein the serial robot includes a stenting unit which includes a support tube, a pre-bent tube mounted within the support tube and a guide wire extending from the support tube for carrying a stent and for piercing a blood vessel.

### CROSS-REFERENCE TO RELATED APPLICATIONS

This application claims the benefit of U.S. Provisional Patent Applications Nos. 61/024,835, filed on Jan. 30, 2008; 61/042,198 filed on Apr. 3, 2008; and 61/046,178 filed on Apr. 18, 2008, which are hereby incorporated by reference herein in their entireties.

### BACKGROUND

Currently several procedures in opthalmology, micro-surgical vasoepidiymostomy, neurosurgery, micro-vascular surgery, and general microsurgery require a dexterous system with the following characteristics: precision and tremor cancellation; dexterity; miniature size suitable for minimally invasive approaches; dual arm operation; ability to insert stents in sub-millimetric blood vessels; ability to deliver funds (e.g. cannulation); ability to perform anastomosis. Currently for most of these types of surgery, micro-stenting procedures can not be performed on sub-millimetric blood vessels in a minimally invasive manner. Stenting procedures are generally applied in cardiovascular procedures where a coronary stent is a small wire mesh tube that is used to help keep coronary (heart) arteries open after angioplasty. A catheter with an empty balloon on its tip is guided into the narrowed part of the artery. The balloon is then filled with air to flatten the plaque against the artery wall. Once the artery is open, a second balloon catheter with a stent on its tip is inserted into the artery and inflated, locking the stent into place.

In ophthalmic surgery it is currently not possible to perform stenting of the blood vessels in the retina in a minimally invasive manner. This task is highly demanding due to the fact that the dimensions of retinal blood vessels being much smaller, around 100-200 microns in diameters, compared to e.g. heart artery and that the eye is an organ which limits the dexterity of the surgical tools quite significantly. The tiny workspace and delicate structures of the eyeball make it currently impossible for surgeons to manipulate several tools simultaneously inside it to do the stenting procedures.

### SUMMARY

Systems, devices and methods related to robot-assisted micro-surgical applications are provided in some embodiments of the disclosed subject matter. The disclosed robot-assisted micro-surgical system allows medical professionals to perform surgery on features that are on the order of microns. This permits surgical procedures that have not been able to be performed in the past, and provide medical professionals with new surgical abilities. In performing micro-surgical procedures, a hybrid robot can be used. This hybrid robot can include a parallel robot and a serial robot. The parallel robot provides positioning of the serial robot over the operative area of the patient. The serial robot can be used to move into the operative area and perform surgical procedures. Given the fine features upon which the robot can be operating, the control system of the hybrid robot may be implemented to enhance the abilities of the medical profession to perform a surgical procedure. This can include force feedback that provides an indication of how the robot is interacting with a patient as well as dexterity enhancements. The dexterity enhancements can react to slight movements in the operative area, stabilize the operative area, and reduce or remove unintended movements of the medical professional controlling the robot. The control of the robot including, for example, the force feedback can provide medical professionals with the ability to operate on micron-sized features.

In some embodiments, a dexterous robotic system for ophthalmic surgery with sufficient dexterity for operation on the retina, including means for stenting and for micro-stenting in micro-vascular surgery, are provided. The robotic system can be implemented with one or more robotic arms. The stenting can be performed on features as small as microns in size. Further, the serial robot can be implemented to provide a stenting unit which can insert a stent in a minimally invasive manner.

### DESCRIPTION OF DRAWINGS

The above and other objects and advantages of the disclosed subject matter will be apparent upon consideration of the following detailed description, taken in conjunction with accompanying drawings, in which like reference characters refer to like parts throughout, and in which:

FIG. 1A illustratively displays a method for using a robot-assisted micro-surgical stenting system in accordance with some embodiments of the disclosed subject matter.

FIG. 1B illustratively displays the general surgical setup for robot-assisted micro-surgical stenting system used on the eye in accordance with some embodiments of the disclosed subject matter.

FIG. 2A illustratively displays a slave dual-arm hybrid-robot positioned over a patient's head in accordance with some embodiments of the disclosed subject matter.

FIG. 2B illustratively displays a slave hybrid-robot with a stenting unit extending from each slave hybrid-robot.

FIG. 3 illustratively displays a robot-assisted micro-surgical stenting system for eye surgery including a tele-robotic master and a slave hybrid-robot in accordance with some embodiments of the disclosed subject matter.

FIG. 4A illustratively displays a slave hybrid-robot illustrating a serial robot and a parallel robot in accordance with some embodiments of the disclosed subject matter.

FIGS. 4B-4D illustratively display a serial connector included in a serial robot in accordance with some embodiments of the disclosed subject matter.

FIGS. 5A-5B illustratively display a serial articulator included in a serial robot in accordance with some embodiments of the disclosed subject matter.

FIGS. 6A-6B illustratively display a stenting unit in accordance with some embodiments of the disclosed subject matter.

FIGS. 6C-6D illustratively display the use of a stenting unit in accordance with some embodiments of the disclosed subject matter.

FIG. 7 illustratively displays a slave hybrid-robot illustrating the legs of a parallel robot in accordance with some embodiments of the disclosed subject matter;

FIGS. 8-9 illustratively display an eye and an i^{th} slave hybrid-robot in accordance with some embodiments of the disclosed subject matter; and

FIGS. 10A-10B illustratively display an organ and an i^{th} slave hybrid-robot in accordance with some embodiments of the disclosed subject matter.

### DETAILED DESCRIPTION

In accordance with the disclosed subject matter, systems, devices, and methods for robot-assisted micro-surgery stenting are disclosed.

The stenting approaches described herein are applied to the minimally invasive micro-surgical arena where the size of the blood vessels or anatomical features are very small (on the order of 5 to 900 microns). While the disclosed subject matter is specifically focused on minimally invasive retinal micro-surgery, this same disclosed subject matter is applicable for general micro-surgical procedures.

In some embodiments, a robot-assisted micro-surgical stenting system includes a tele-robotic microsurgical system and a micro-stenting unit. The tele-robotic microsurgical system can have a slave hybrid robot having at least two robotic arms (each robotic arm having a serial robot attached to a parallel robot) and a tele-robotic master having at least two user controlled master slave interfaces (e.g., joysticks). Further, the micro-stenting unit is connected to the serial robot for each robotic arm and includes a tube housing a pre-bent superelastic NiTi (Nickel Titanium) cannula that is substantially straight when in the support tube. The stent is carried on the NiTi (superelastic Nickel Titanium) guide wire using each of the user controlled master slave interfaces, the user can control movement of the at least two robotic arms by controlling the parallel robot and serial robot for each robotic arm. That is, the user can control the combined motion of the serial robot and parallel robot for each arm by the master slave interfaces. The cannula and the guide wire can be manufactured using superelastic Nickel Titanium in some embodiments.

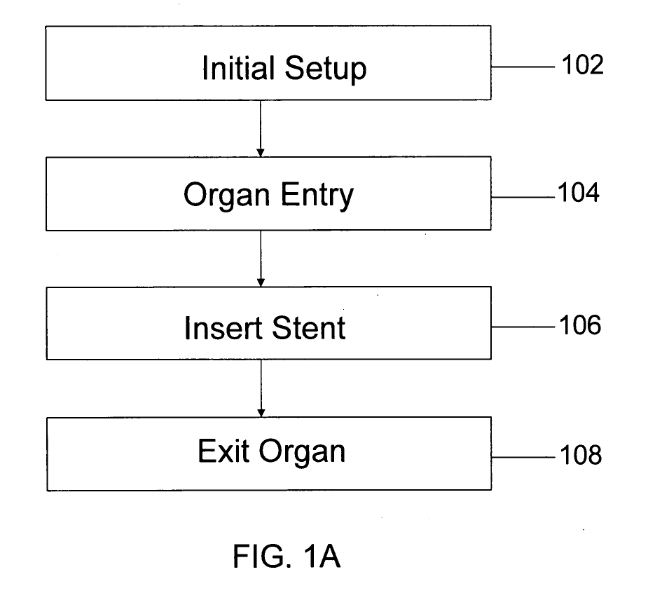

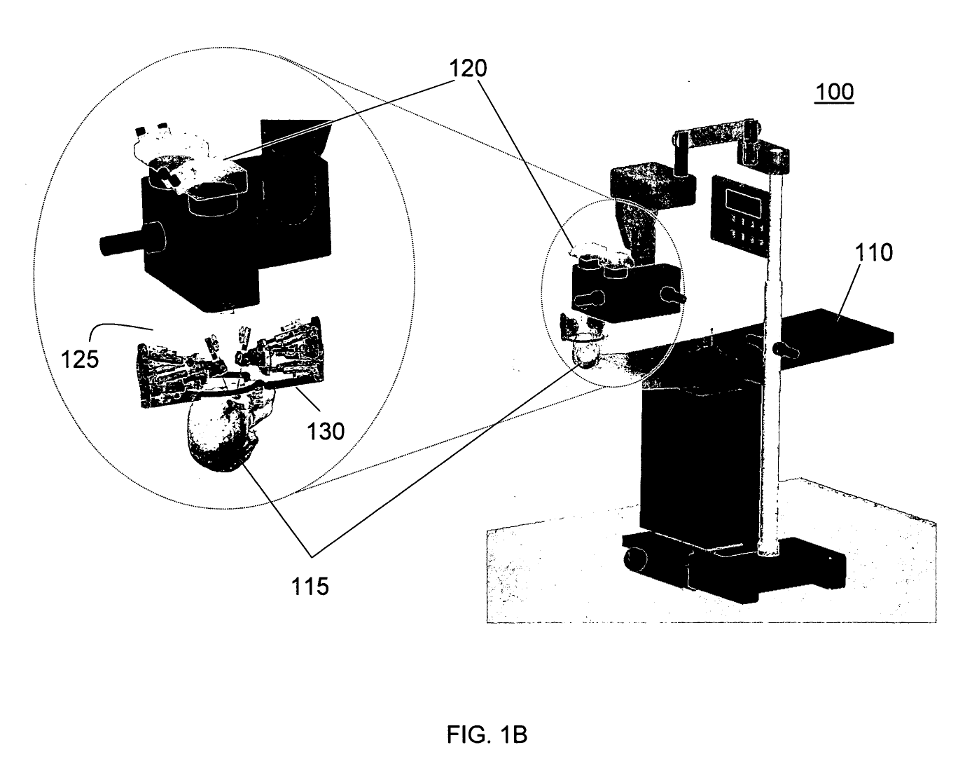

Referring to FIG. 1B, the general surgical setup for robot-assisted micro-surgical stenting on the eye is displayed. In some embodiments, a general surgical setup for eye surgery 100 includes a surgical bed 110, a surgical microscope 120, a slave hybrid-robot 125, and a tele-robotic master (not shown). The patient lies on surgical bed 110, with his head 115 positioned as shown. During eye surgery a patient located on surgical bed 110, has a frame 130 releasably attached to their head, and a slave hybrid-robot releasably attached to frame 130. Further, a medical professional views the operative area through surgical microscope 120 and can control the slave hybrid-robot 125. This control can include insertion of a stent, drug delivery, aspiration, light delivery, and delivery of at least one of microgrippers, picks, and micro knives. The control of slave can be through the tele-robotic master which is in communication with slave hybrid-robot 125.

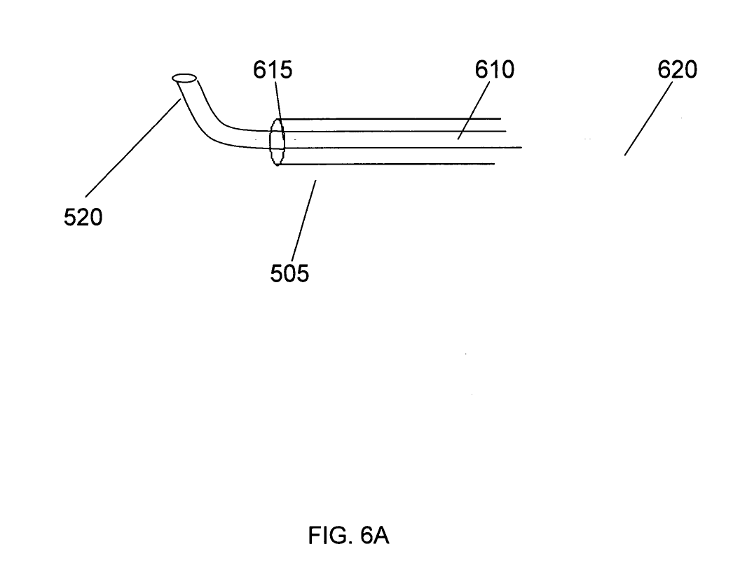

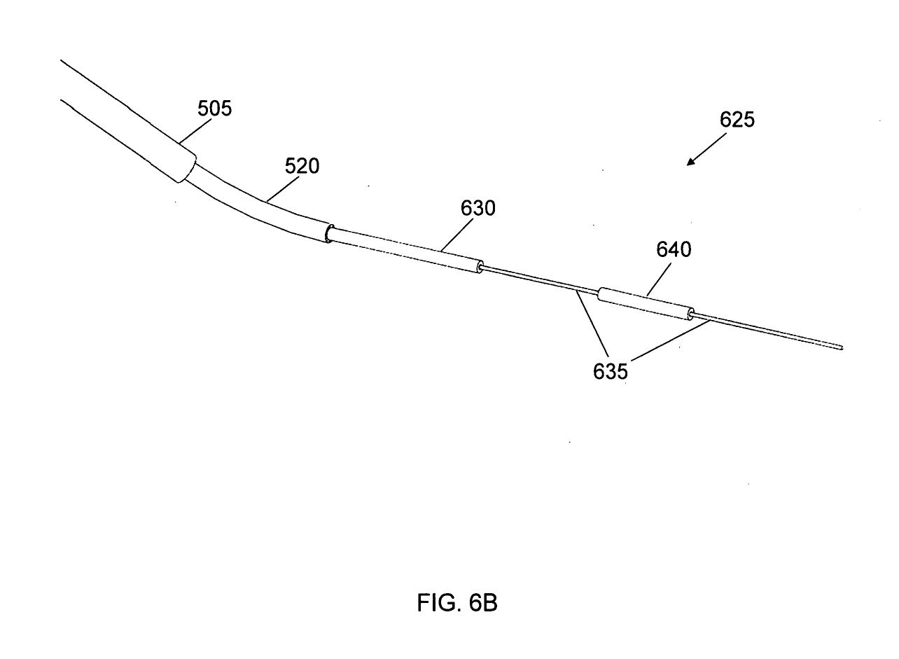

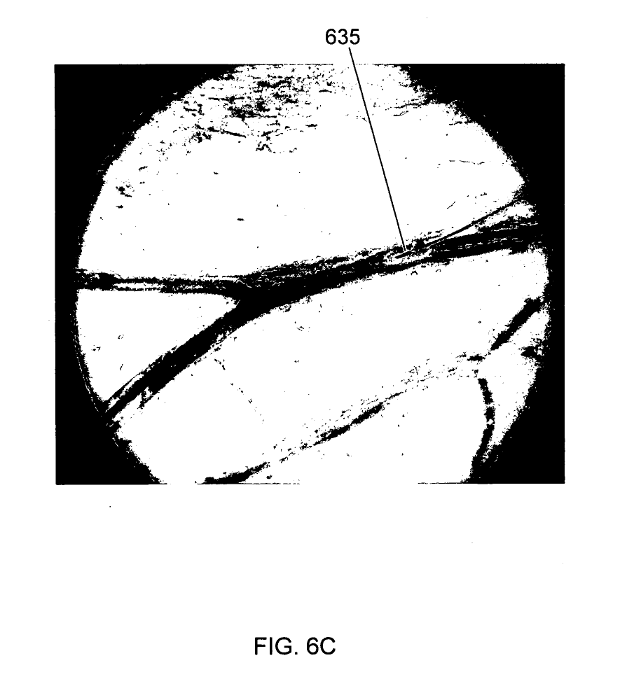

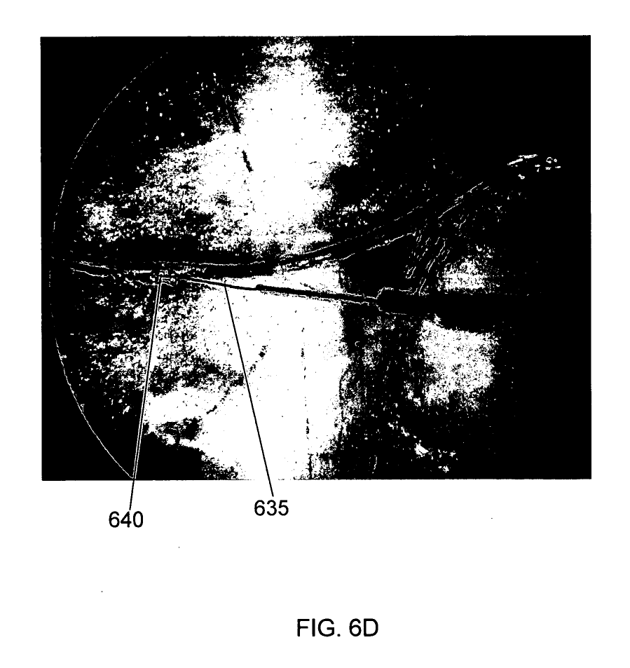

Referring to FIG. 1A a method for using a robot-assisted micro-surgical stenting system is illustratively displayed. For initial setup (102 in FIG. 1A), the slave-hybrid robot can be positioned over the organ (e.g., attached to a frame connected to the head of a patient when the organ is the eye). For example, a slave-hybrid robot having a first robotic arm (having a first parallel robot and first serial robot) and a second robotic arm (having a second parallel robot and a second serial robot) can have both arms in a position minimizing the amount of movement needed to enter the organ. For organ entry (104 in FIG. 1A), using a first user controlled master slave interface to control the first robotic arm, the user can insert a first support tube 505 (See FIGS. 6A-6B), housing a pre-bent tube 520, guide wire 635 (FIG. 6B) and stent, into a patient's organ by moving the first parallel robot. Similarly, using a second user controlled master slave interface to control the second robotic arm, the user can insert a second tube into the patient's organ by moving the second parallel robot. Once inside the organ, the user can insert the stent (106 in FIG. 1A),

For inserting the stent inside the organ (106 in FIG. 1A), using the first user controlled master slave interface to control the first robotic arm, the user can control the first serial robot extending the first pre-bent tube 520 and guide wire 635 out of the first supporting tube 505, the first pre-bent tube 520 bending as it exits the first supporting tube 505. This bending represents one degree of freedom for the serial robot as described below. Further, using the first user controlled master slave interface to control the first robotic arm, the user can use the first serial robot to rotate at least one of the first pre-bent tube 520 and the first support tube 505 about their longitudinal axis (hence positioning the stent guide wire inside the organ). This rotation about the longitudinal axis represents a second degree of freedom for the serial robot. Similarly, using the second user controlled master slave interface to control the second robotic arm, the user can use the second serial robot to move a second pre-bent tube out of the second support tube. The second pre-bent tube bends as it exits the second support tube. Again, similarly, the user can rotate at least one of the second pre-bent tube and the second support tube about their longitudinal axis. In some instances, delivering a second pre-bent tube out of a second support tube is not necessary.

For exiting the organ (106 in FIG. 1A), that is, to remove the support tube 505, pre-bent tube 520 and guide wire 635 from the organ, the user uses the first, user controlled master slave interface to control the first robotic arm. The user retracts the first guide wire 635 and tube 630 until both exit the blood vessel. The user then uses the hybrid robot to move the tip of the stenting unit away from the retina in order to allow safe retraction of the pre-bent tube 520 into the first support tube 505 using the first serial robot. Using both the first user controlled master slave interface to control the first robotic arm, the user can move the first parallel robot to retract the first support tube 505 from the organ. In cases of emergency, the serial robot can be removed from the eye by releasing a fast clamping mechanism connecting it to a parallel robot, and subsequently removing the frame with the two parallel robots.

It will be apparent that the disclosed subject matter can be used for inserting stents in any organ in the body. For ease in understanding the subject matter presented herein, the following description focuses on the insertion of micro-surgical stents in the eye.

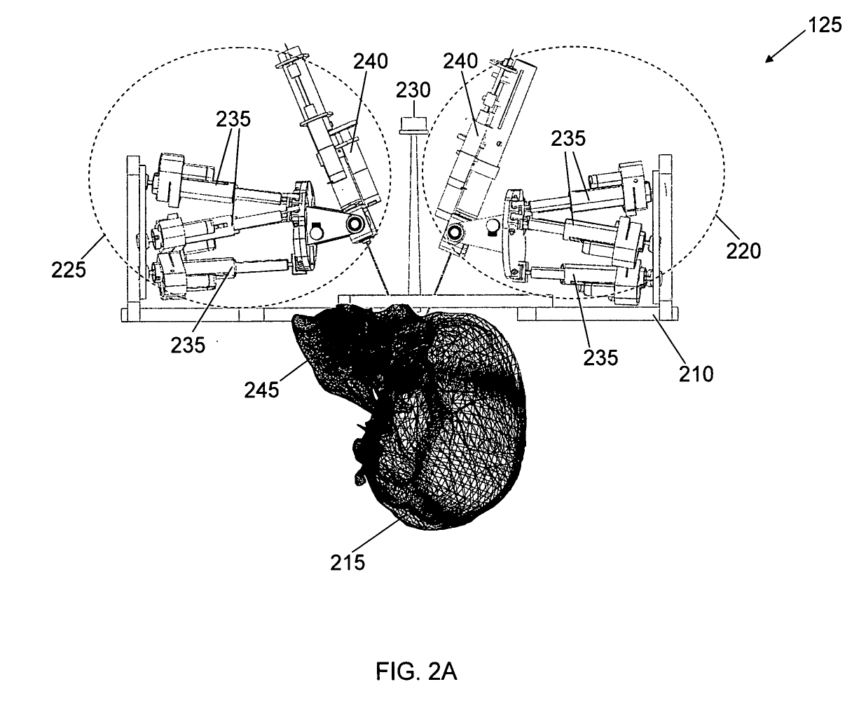

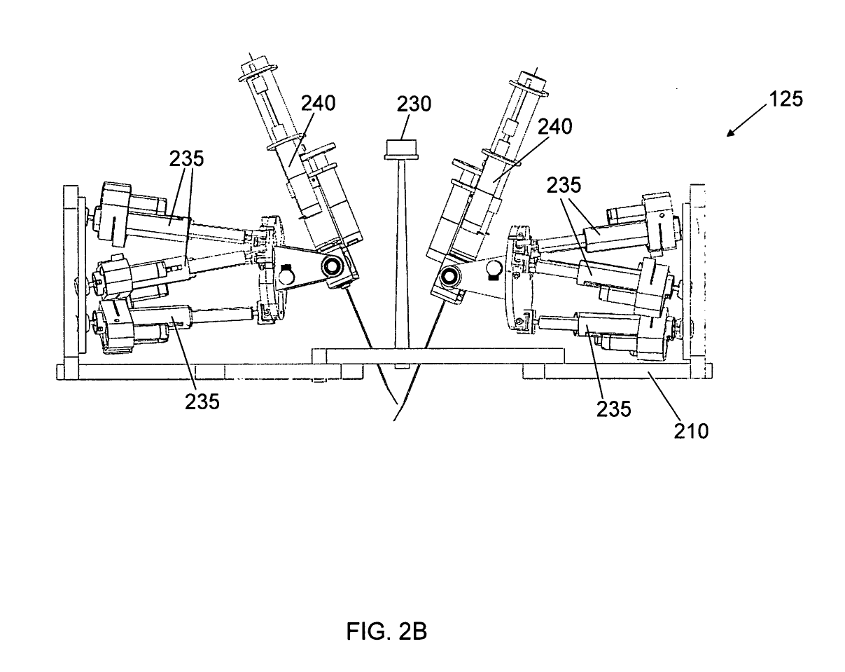

Referring to FIG. 2A, a slave hybrid-robot 125 positioned over a patient's head is displayed. In some embodiments, the slave hybrid-robot 125 can be attached to a frame 210 which in turn is attached to a patient's head 215. Further, slave hybrid-robot 125 includes a first robotic arm 220 and a second robotic arm 225 that can be attached to frame 210 in a manner that does not intersect the microscope view cone 230. The microscope may be attached to a camera to allow projection of pictures or video to a screen. Still further, in some embodiments, first robotic arm 220 and second robotic arm 225 can include a parallel robot 235 (e.g., a Stewart platform, Stewart/Gough platform, delta robot, etc.) and a serial robot 240 (e.g., a robot consisting of a number of rigid links connected with joints). Some parts of the first and second robotic arms can be permanently attached to the frame while other parts can be releasably attached to the frame. Further, the serial robot can be releasably attached to the parallel robot. For example, for a robotic arm including a parallel and a serial robot, the parallel robot can be permanently attached to the frame and the serial robot can be releasably attached to the parallel robot. In some embodiments, the serial robot can be releasably attached to the parallel robot by, for example, lockable adjustable jaws.

In some embodiments, the slave hybrid-robot includes at least two robot arms releasably attached to the frame. For example, the robot arms can be attached to the frame by an adjustable lockable link, a friction fit, a clamp fit, a screw fit, or any other mechanical method and apparatus deemed suitable. Further, the robotic arms can be permanently attached to the frame. For example, the robotic arms can be attached by welding, adhesive, or any other mechanism deemed suitable.

In some embodiments, first robotic arm 220 and second robotic arm 225 can be adjusted into location at initial setup of the system (e.g., at the beginning of surgery). This can be done, for example, to align the robotic arms with the eye. Further, first robotic arm 220 and second robotic arm 225 can have a serial robot and a parallel robot where only one of the serial robot or parallel robot can be adjusted into location at initial setup of the system.

In some embodiments, frame 210 can be attached to the patient's head by a bite plate 245 (e.g., an item placed in the patient's mouth which the patient bites down on) and a surgical strap 250. Frame 210 can be designed to produce the least amount of trauma to a patient when attached. For example, frame 210 can be attached to a patient's head by a coronal strap (e.g., a strap placed around the patient's head) and a locking bite plate (e.g., a bite plate which can be locked onto the patient's mouth where the bite plate locks on the upper teeth). Any mechanism for attaching the frame to the patient's head can be used. For example, the frame can be attached to the patient's head by a compression mechanism that uses compression to hold the frame affixed or an attachment piece. The compression mechanism can be a belt or clamp and the attachment piece can removeably attach to a part of the patient.

Further, bite plate 245 can include air and suction access (not shown). For example, in an emergency, first robotic arm 220 and second robotic arm 225 can be released from the frame and the patient can receive air and suction through tubes (not shown) in the bite plate access.

Frame 210 can be made using a substantially monolithic material constructed in a substantially circular shape with a hollow center. Further, the shape of frame 210 can be designed to fit the curvature of the patient's face. For example, the frame 210 can be substantially round, oval, or any other shape deemed suitable. The frame material can be selected to be fully autoclaved. For example, the frame material can include a metal, a plastic, a blend, or any other material deemed suitable for an autoclave. Further still, frame 210 can include a material that is not selected to be fully autoclaved. That is, the frame can be for one time use.

In some embodiments, first robotic arm 220 and second robotic arm 225 include hybrid-robots. It will be understood that a hybrid-robot refers to any combination of more than one robot combined for use on each of the robotic arms. For example, in some embodiments, first robotic arm 220 and second robotic arm 225 include a six degree of freedom parallel robot (e.g., a Stewart platform, Stewart/Gough platform, delta robot, etc.) attached to a two degree of freedom serial robot (e.g., an intra-ocular dexterity robot) which when combined produce 16 degrees of freedom in the system. The hybrid-robots can include a parallel robot with any number of degrees of freedom. Further, the two degree of freedom serial robot (e.g., intra-ocular dexterity robot) can provide intra-ocular dexterity while the parallel robot can provide global high precision positioning of the eye and the stent inside the eye. Still further, the hybrid-robots can include any combination of robots including a serial robot, parallel robot, snake robot, mechanatronic robot, or any other robot deemed suitable.

First robotic arm 220 and second robotic arm 225 can be substantially identical. For example, both first robotic arm 220 and second robotic arm 225 can include a parallel robot and a serial robot. Further, first robotic arm 220 and second robotic arm 225 can be substantially different. For example, first robotic arm 220 can include a first parallel robot attached to a second rigid cannula for suction.

In some embodiments, slave hybrid-robot 125 includes only two robotic arms. Using two robotic arms increases the bimanual dexterity of the user. For example, the two robotic arms can be controlled by a medical professional using two user controlled master slave interfaces (e.g., one controller in contact with each hand). Further, more than two robotic arms can be used in slave hybrid-robot 125. For example, three robotic arms can be used in slave hybrid-robot 125. Any suitable number of robotic arms can be used in slave hybrid-robot 125.

The robotic arms can be constructed to be reused in future operations. For example, first robotic arm 220 and second robotic arm 225 can be designed to be placed in an autoclave. Further, first robotic arm 220 and second robotic arm 225 can be designed to allow the use of sterile drape. Still further, parts of the robotic arms can be designed for one time use while other parts can be designed to be used in future operations. For example, first robotic arm 220 and second robotic arm 225 can include a disposable cannula, which can be used one time, and a reusable parallel robot.

In some embodiments, the slave hybrid-robot can be designed to use less than 24 Volts and 0.8 Amps for each electrical component. Using less than 24 Volts and 0.8 Amps can minimize safety concerns for the patient. Further, in some embodiments, both the parallel robot and serial robot allow sterile draping and the frame supporting the parallel and serial robot can be designed to be autoclaved.

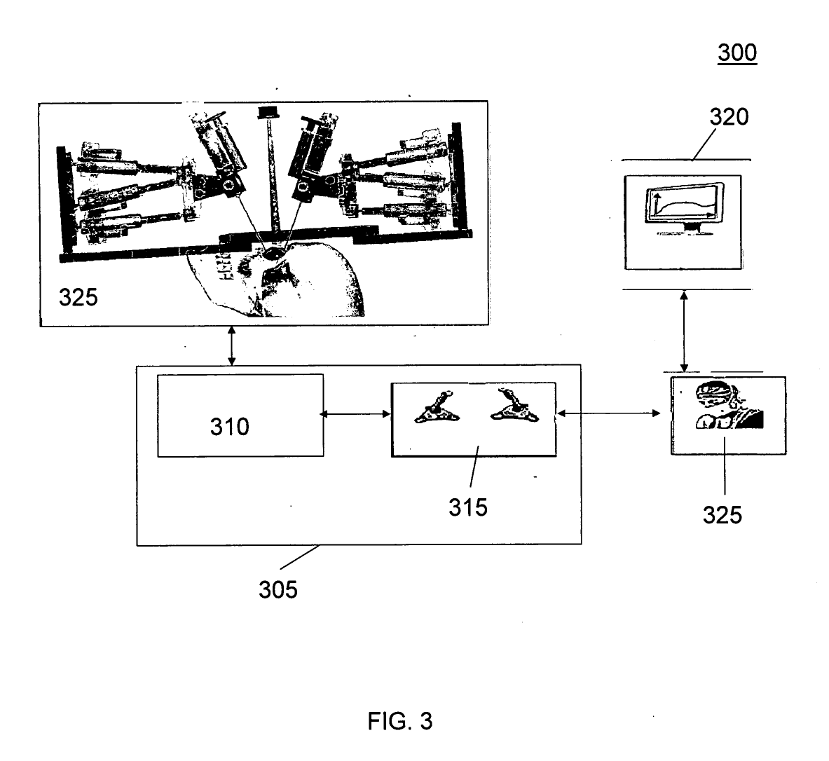

Referring to FIG. 3, in some embodiments, a robot-assisted microsurgical stenting system for eye surgery 300 includes a tele-robotic master 305 and a slave hybrid-robot 325. In some embodiments, tele-robotic robotic master 305 includes a controller 310 and a user controlled master slave interface 315 (e.g., two force feedback joysticks). In some embodiments, controller 310 includes at least one of a dexterity optimizer, a force feedback system, and a tremor filtering system.

The force feedback system can include a display 320 for indicating to a medical professional 325 the amount of force exerted by the robotic arms (e.g., the force on the cannula in the eye). Further, the force feedback system can include providing resistance on user controlled master slave interface 315 as the medical professional increases force on the robotic arms. Further still, at least one of the robotic arms can include a force sensor and torque sensor to measure the amount of force or torque on the arms during surgery. These sensors can be used to provide force feedback to the medical professional. Forces on the robotic arms can be measured to prevent injuring patients. The forces that the robot applies on the access port in the eye may be measured, for example, by using a six-axis load cell located in the interface between component 406 and the serial robot 240. The intra-ocular forces applied by the serial robot on the retina may be measured by a number of different techniques, including using a micro-electro-mechanical force sensor (e.g. miniature capacitive PZT sensor), or by visual tracking of the deflection of the stent wire 635.

A tremor reducing system can be included in robotic master 305. For example, tremor reduction can be accomplished by filtering the tremor of the surgeon on the tele-robotic master side before delivering motion commands. For example, the motions of a master slave interface (e.g., joystick) can be filtered and delivered by the controller as set points for a PID (proportional, integral, and differential) controller of the slave hybrid-robot. In this example the two tilting angles of the master joystick can be correlated to axial translations in the x- and y directions. The direction of the master slave interface (e.g., joystick) can be correlated to the direction of movement of the slave in the x-y plane while the magnitudes of tilting of the master slave interface (e.g., joystick) can be correlated to the magnitude of the movement velocity of the robotic slave in x-y plane. In another embodiment the user can control the slave hybrid robot by directly applying forces to a tube (described below) included in the serial robot. Further, the serial robot can be connected to the parallel robot through a six-axis force and moment sensor that reads forces that the user applies and can deliver signals to the controller 310 that translates these commands to motion commands while filtering the tremor of the hand of the surgeon. Any suitable method for tremor reducing can be included in tele-robotic master 305. For example, any suitable cooperative manipulation method for tremor reducing can be used.

The controller 310 can be used to control the movements of the robot, which can include the positioning and actions performed by the robot. The controller can receive these commands through a communications channel such as a copper based wire (e.g., an Ethernet wire). The controller can be a microprocessor with a computer readable medium, a programmable logic controller, an application specific integrated circuit, or any other applicable device. The controller 310 can perform calculations as described below to determine how the robot moves. The controller 310 can also receive information from sensors on the parallel and serial robots and use this information in performing the calculations to determine the robot's movement.

In some embodiments, a dexterity optimizer can include any mechanism for increasing the dexterity of the user. For example, the dexterity optimizer can utilize a preplanned path for entry into the eye. In some embodiments, the dexterity optimizer takes over the delivery of the tube into the eye by using the preplanned path. In some embodiments a dexterity optimizer can constrain hand movements. In some embodiments a dexterity optimizer can give cues for movements to the user.

The tele-robotic master and slave hybrid-robot can communicate over a high-speed dedicated Ethernet connection. Any communications mechanism between the tele-robotic master and slave hybrid-robot deemed suitable can be used. Further, the medical professional and the tele-robotic master can be in a substantially different location than the slave hybrid-robot and patient.

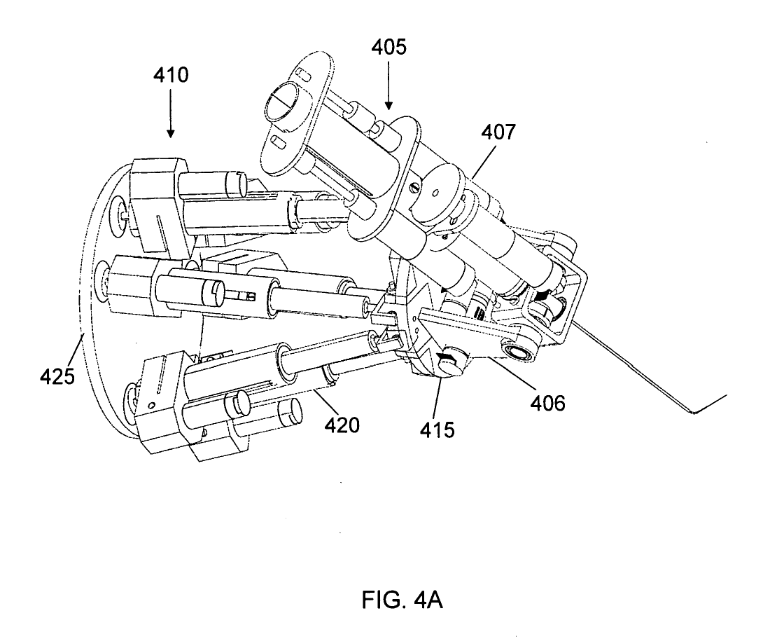

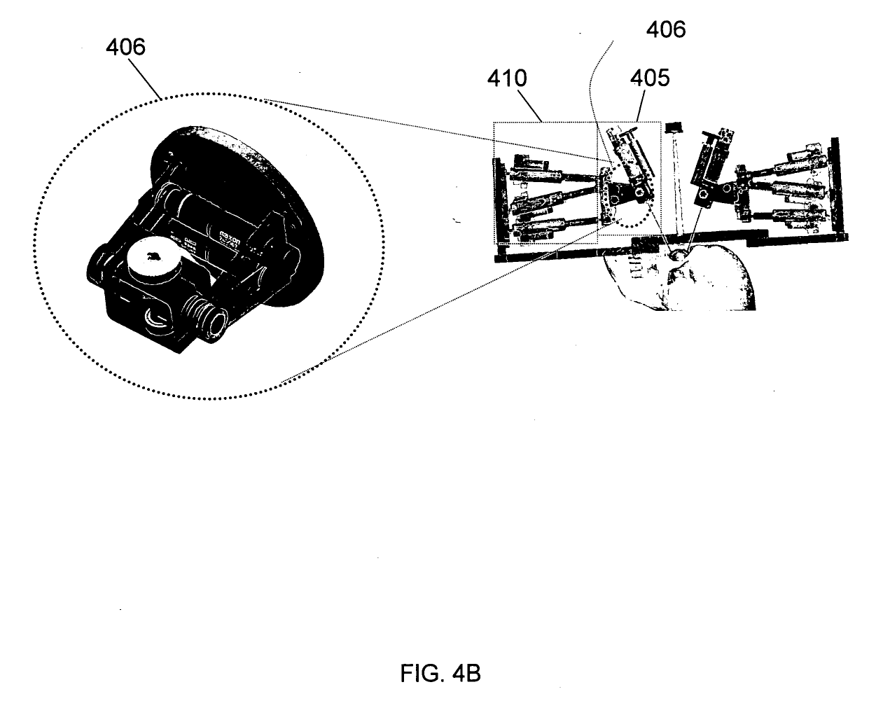

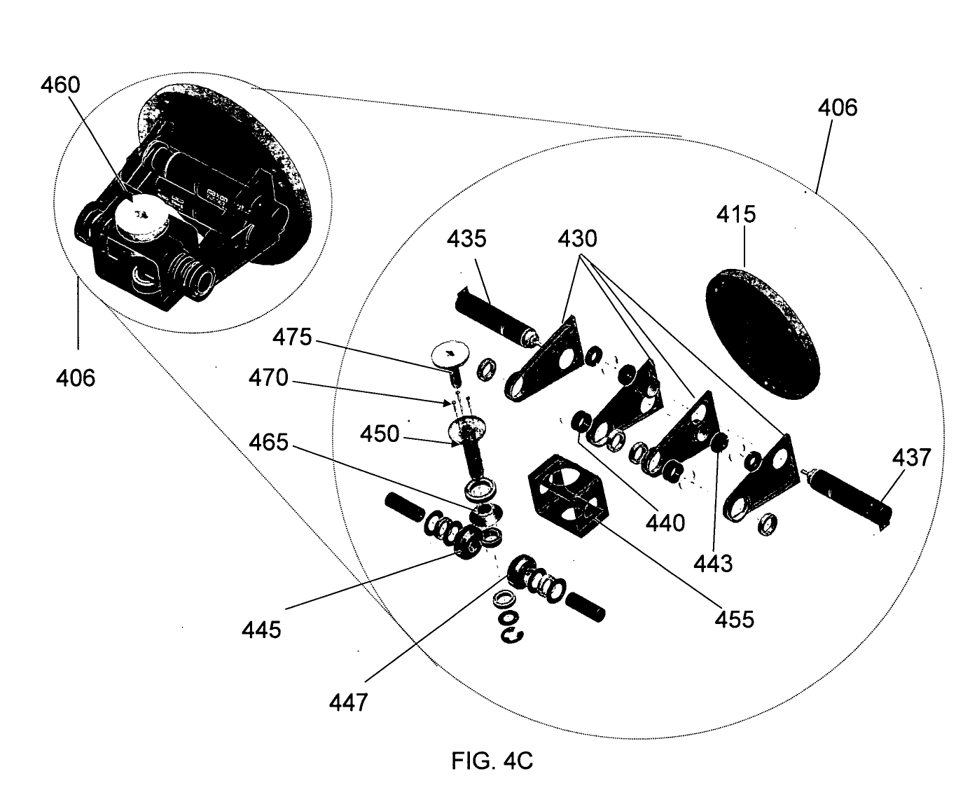

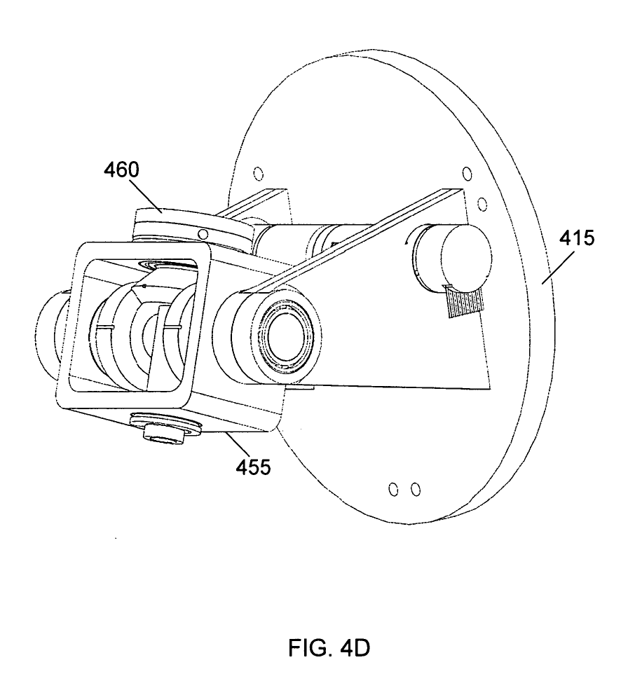

Referring to FIG. 4A, in some embodiments, the slave hybrid-robot can include a serial robot 405 and a parallel robot 410. Further, in some embodiments, serial robot 405 can include a serial connector 406 for connecting a platform 415 (e.g., the parallel robot's platform) and a serial articulator 407. Any mechanical connection can be used for connecting the parallel robot's platform and serial articulator 407. Platform 415 can be connected to legs 420 which are attached to base 425.

Referring to FIG. 4B, a serial robot 405 including serial connector 406 is illustratively displayed. The serial connector 406 is enlarged to provide a clearer view of the serial connector. Referring to FIG. 4C, an exploded view of serial connector 406 is displayed for a clearer view of a possible construction for serial connector 406. Any suitable construction for serial connector 406 can be used. For example, serial connector 406 can connect serial articulator 407 (FIG. 4A) with parallel robot 410 (FIG. 4A). Referring to FIG. 4C, platform 415 (e.g., the parallel robot moving platform) can support hollow arms 430 that can support a first electrical motor 435 and a second electric motor 437. First electric motor 435 and second electric motor 437 can actuate a first capstan 440 and a second capstan 443 via a first wire drive that actuate anti-backlash bevel gear 445 and a second wire drive actuate anti-backlash bevel gear 447 that can differentially actuate a third bevel gear 465 about its axis and tilt a supporting bracket 455. Differentially driving first electric motor 435 and second electric motor 437, the tilting of bracket 455 and the rotation of a fast clamp 460 about the axis of the cannula can be controlled.

Further referring to FIG. 4C, an exploded view of the fast clamp 460 is displayed for a clearer view of a possible construction for fast clamp 460. Fast clamp 460, included in serial connector 406, can be used to clamp instruments that are inserted through the fast clamp 460. Any suitable construction for fast clamp 460 can be used. For example, fast clamp 460 can include a collet housing 450, connecting screws 470, and a flexible collet 475. Connecting screws 470 can connect collet housing 450 to third bevel gear 465. Collet housing 450 can have a tapered bore such that when flexible collet 475 is screwed into a matching thread in the collet housing 450 a flexible tip (included in flexible collet 475) can be axially driven along the axis of the tapered bore, hence reducing the diameter of the flexible collet 475. This can be done, for example, to clamp instruments that are inserted through the fast clamp 460. Any other suitable mechanism for clamping instruments can be used.

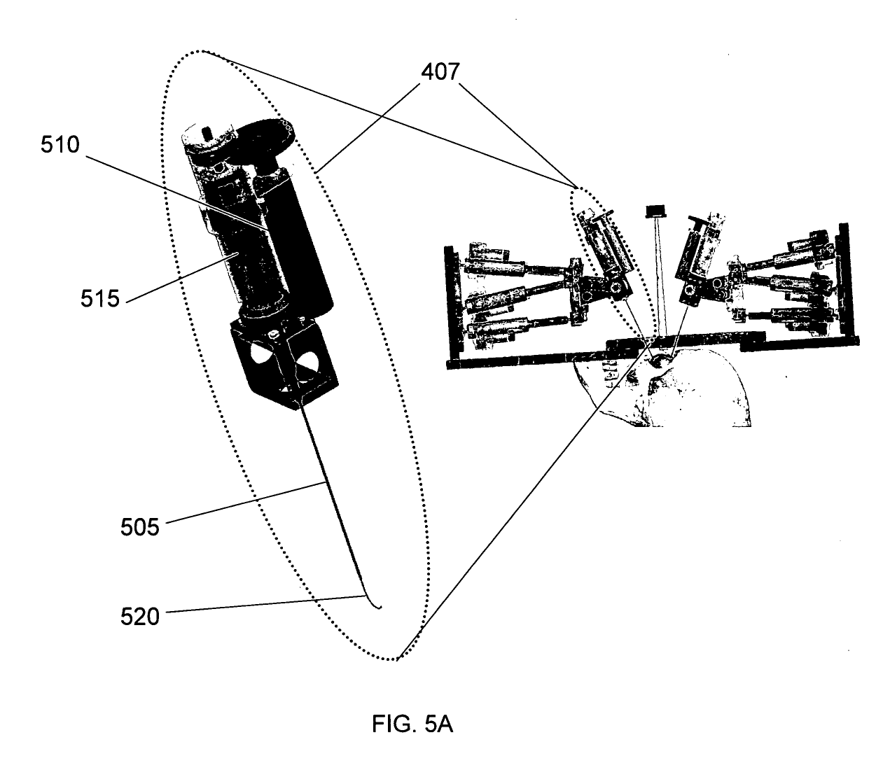

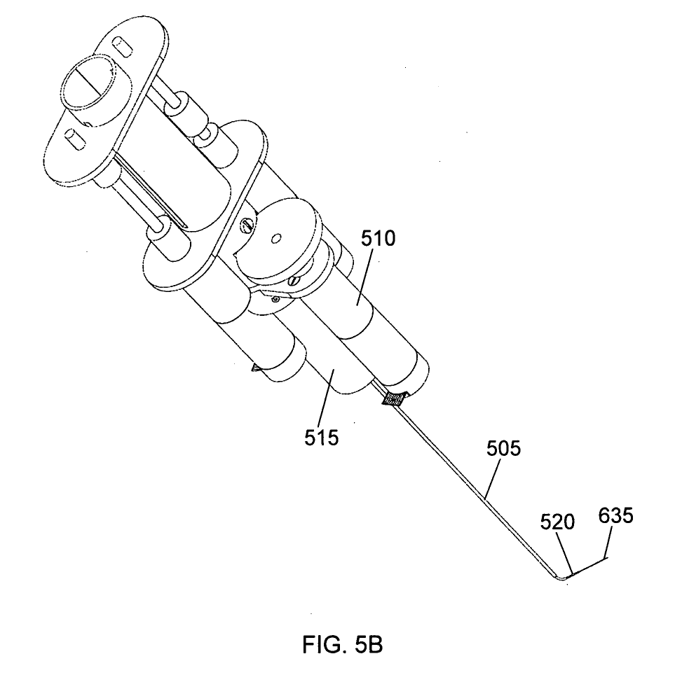

Referring to FIGS. 5A-5B, in some embodiments, the serial robot includes a serial articulator 407 for delivering at least one of a support tube 505 and a cannula or pre-bent tube 520 into the eye. In some embodiments, for example, serial robot articulator 407 includes a servo motor 510 and high precision ball screw 515 for controlling delivery of at least one of support tube 505 and pre-bent tube 520 housing a guide wire 635 (FIG. 5B). Servo motor 510, coupled to high-precision ball screw 515, can add a degree of freedom to the system that can be used for controlling the position of pre-bent tube 520 with respect to support tube 505. For example, servo motor 510 can be coupled to a hollow lead screw (not shown) that when rotated drives a nut (not shown) axially. Further, for example, pre-bent tube 520 can be connected to the nut and move up/down as servo motor 510 rotates the lead screw (not shown). Any suitable mechanism for controlling the delivery of support tube 505 and pre-bent tube 520 can be used. Further, in some embodiments, support tube 505 houses pre-bent tube 520.

Referring to FIGS. 6A-6B, in some embodiments, pre-bent tube 520, stent pushing tube 630, guide wire 635 and stent 640 can be delivered through support tube 505 into the eye. FIG. 6A illustratively displays a pre-bent tube 520 after exiting support tube 505 (hence the pre-bent tube 520 has assumed its pre-bent shape). The pre-bent shape of pre-bent tube 520 can be created by using any superelastic shape memory alloy (e.g., NiTi) and setting the shape so that the cannula assumes the bent position at a given temperature (e.g., body temperature, room temperature, etc.). Further, although pre-bent tube 520 is described as having a specific pre-bent shape, any shape deemed suitable can be used (e.g., s-shaped, curved, etc.). Support tube 505 can include a proximal end 610 and a distal end 615. Further, pre-bent tube 520 can exit distal end 615 of support tube 505. Tube 505 and pre-bent tube 520 can be constructed of different suitable materials, such as a plastic (e.g, Teflon, Nylon, etc), metal (e.g, Stainless Steel, NiTi, etc), or any other suitable material. Further, in some embodiments, at least one of tube support tube 505 and pre-bent tube 520 can rotate about longitudinal axis 620.

Further, in some embodiments, pre-bent tube 520 can be a backlash-free super-elastic NiTi cannula to provide high precision dexterous manipulation. Using a backlash-free super-elastic NiTi cannula increases the control of delivery into the orbit of the eye by eliminating unwanted movement of the cannula (e.g., backlash). Further, the bending of cannula 520 when exiting tube 505 can increase positioning capabilities for insertion of the stent 640.

Referring to FIG. 6B the stenting unit actuates two concentric NiTi tubes 505, 520 and one NiTi guide wire 635. Each tube/wire can be actuated independently. So each unit of the robot has 3 DoF's (Degrees of Freedom).

The stent 640 is a sharpened (or bevel cut) micro-tube that is carried on a NiTi wire 635 sharp enough to pierce into a blood vessel. The support tube 505 is fixed and not actuated. It serves as the support of all inner tubes and wires. In an ophthalmic surgery this tube is inserted through the sclera. The pre-bent tube 520 can be created under heat treatment. The distal end of the pre-bent tube 520 assume the predetermined shape as the tube is extended out of the support tube 505.

The stent pushing tube 630 serves to push the stent 640 into the blood vessel. The blood vessel poking wire 635, serves double duties as the needle to poke into the blood vessel as well as the guide wire to accurately position the stent 640. Once the stent 640 is put in position, the wire will be retracted and leave the stent in the blood vessel. This action is coordinated with control of the stent pushing tube 630 that keeps the stent 640 at the desired position in the blood vessel. In some embodiments, the stent 640 has a micro-machined screw-like external helix. In such case, the stent 640 is inserted into the blood vessel mounted on the guide wire 635 through a prismatic connection that allows delivery of torque. By rotating the guide wire 635 the stent 640 advances along the guide wire to the derired position in the blood vessel. The guide wire 635 is subsequently pulled out of the stent and the blood vessel.

Referring to FIG. 6C, the guide wire 635 is shown piercing a blood vessel, and FIG. 6D shows a stent 640 inserted in a blood vessel.

The sizes of the tubes and wire can be any size suitable to be inserted in the applicable blood vessel. In some embodiments, the support tube 505 can be a diameter of approximately 0.90 mm, the pre-bent tube 520 can be a diameter of 0.55 mm; the stent pushing tube 630 can have an inner diameter of 0.1 mm and outer diameter of 0.2 mm; and the stent 640 can also have an inner diameter 0.1 mm and outer diameter 0.2 mm. The guide wire 635 can be a diameter of 75 microns. In some embodiments, the stent 640 has an interior diameter of 50 microns and an outer diameter of up to 150. In such a case the guide wire 635 would have a diameter of less than 50 microns.

A power generator is used to provide voltage to the joystick 315. The joysticks are under velocity control, meaning that the further the joystick is tilted from the central position, the larger speed of the actuators is expected. At the central position of the joystick, the positions of the motors are fixed by using the closed-loop control from the encoders. This control scheme is that the user serves as the feedback provider by looking at the robot for the target point and determining how much he/she should tilt the joystick. Once in position, the joystick is just tilted back to the central position so that the motor is accurately fixed in position due to the closed-loop system.

The microscope 230 is used to provide clearer view of the surgery. A light source provides additional lighting for the microscope 230. The platform provides the adjustment of the height of the experimented membranes.

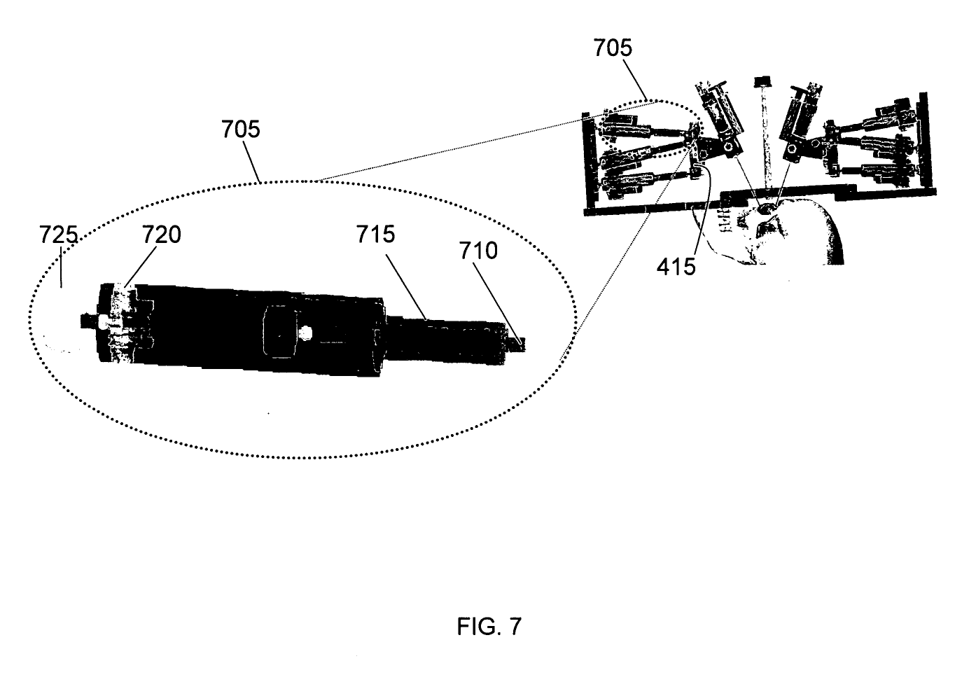

Referring to FIG. 7, the parallel robot can include a plurality of independently actuated legs 705. As the lengths of the independently actuated legs are changed the position and orientation of the platform 415 changes. Legs 705 can include a universal joint 710, a high precision ball screw 715, anti-backlash gear pair 720, and a ball joint 725. The parallel robot can include any number of legs 705. For example, the parallel robot can include three to six legs.

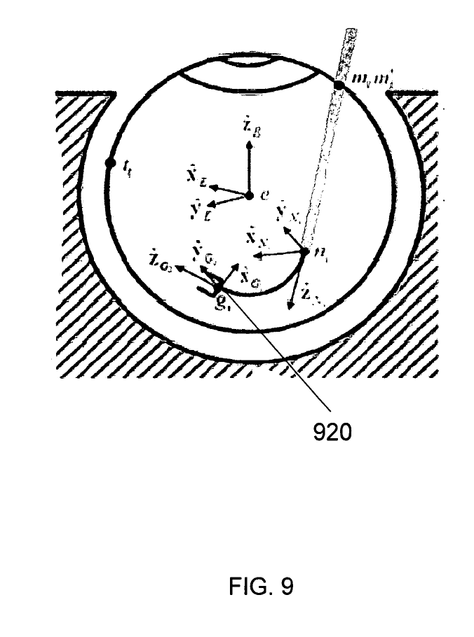

In some embodiments, a unified kinematic model accounts for the relationship between joint speeds (e.g., the speed at which moving parts of the parallel and serial robots translate and rotate) of the two robotic arms of the slave hybrid-robot, and twist of the eye and the movements of the components of the stenting unit inside the eye. It will be understood that the twist relates to the six dimensional vector of linear velocity and angular velocity where the linear velocity precedes the angular velocity. The twist can be required to represent the motion of an end effector, described below (920 in FIG. 9). Further, this definition can be different from the standard nomenclature where the angular velocity precedes the linear velocity (in its vector presentation).

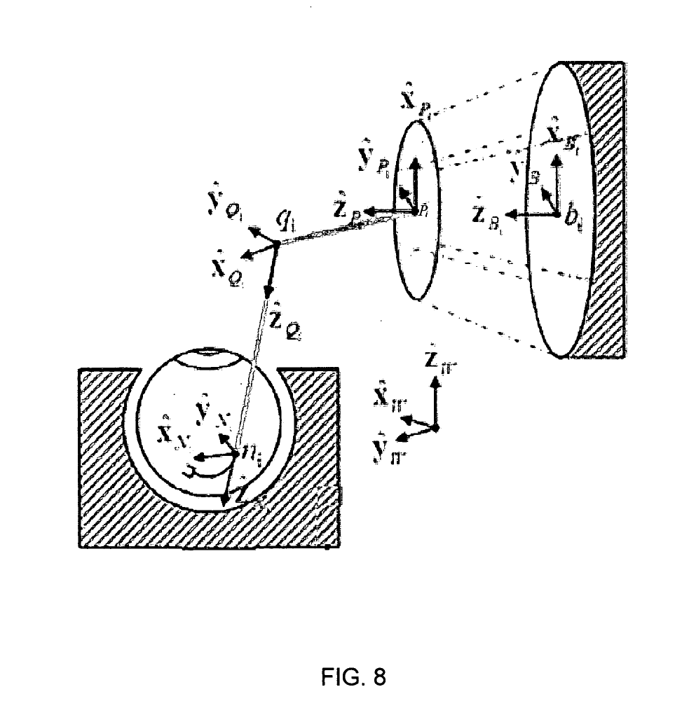

Referring to FIG. 8, the eye and an i^{th} hybrid robot is displayed. The eye system can be enlarged, FIG. 9, for a clearer view of the end effector (e.g., the device at the end of a robotic arm designed to interact with the environment of the eye, such as the pre-bent tube or the guide wire delivered through the pre-bent tube) and the eye coordinate frames. The coordinate system can be defined to assist in the derivation of the system kinematics. For example, the coordinate systems described below are defined to assist in the derivation of the system kinematics. The world coordinate system {W} (having coordinates \hat{x}_{W}, ŷ_{W}, \hat{z}_{W}) can be centered at an arbitrarily predetermined point in the patient's forehead with the patient in a supine position. The \hat{z}_{W} axis points vertically and ŷ_{W} axis points superiorly (e.g., pointing in the direction of the patients head as viewed from the center of the body along a line parallel to the line formed by the bregma and center point of the foramen magnum of the skull). A parallel robot base coordinate system {Bᵢ} of the i^{th} hybrid robot (having coordinates \hat{x}_{B}_{ᵢ}, ŷ_{B}_{ᵢ}, \hat{z}_{B}_{ᵢ}) can be located at point bᵢ (i.e., the center of the platform base) such that the \hat{z}_{B}_{ᵢ} axis lies perpendicular to the platform base of the parallel robot base and the \hat{x}_{B}_{ᵢ} axis lies parallel to \hat{z}_{W}. The moving platform coordinate system of the i^{th} hybrid robot {Pᵢ} (having coordinates \hat{x}_{P}_{ᵢ}, ŷ_{P}_{ᵢ}, \hat{z}_{P}_{ᵢ}) lies in center of the moving platform, at point pᵢ, such that the axes lie parallel to {Bᵢ} when the parallel platform lies in a home configuration. A parallel extension arm coordinate system of the i^{th} hybrid {Qᵢ} (having coordinates \hat{x}_{Q}_{ᵢ}, ŷ_{Q}_{ᵢ}, \hat{z}_{Q}_{ᵢ}) can be attached to the distal end of the arm at point qᵢ, with \hat{z}_{Q}_{ᵢ} lying along the direction of the insertion guide wire of the robot, in vector direction \vec{qᵢnᵢ}, and \hat{x}_{Q}_{ᵢ} being fixed during setup of the stenting procedure. The serial robot base coordinate system of the i^{th} hybrid robot {Nᵢ} (having coordinates \hat{x}_{N}_{ᵢ} ŷ_{N}_{ᵢ} \hat{z}_{N}_{ᵢ}) lies at point nᵢ with the \hat{z}_{N}_{ᵢ}, axis also pointing along the insertion needle length of vector \vec{qᵢnᵢ} and the ŷ_{N}_{ᵢ} axis rotated from ŷ_{Q}_{ᵢ} an angle q_{S}_{ᵢ}₁ about \hat{z}_{N}_{ᵢ}. The end effector coordinator system {Gᵢ} (having coordinates \hat{x}_{G}_{ᵢ}, ŷ_{G}_{ᵢ}, \hat{z}_{G}_{ᵢ}) lies at point gᵢ with the \hat{z}_{G}_{ᵢ} axis pointing in the direction of the end effector gripper 920 and the ŷ_{G}_{ᵢ} can be parallel to the ŷ_{N}_{ᵢ} axis. The eye coordinate system {E} (having coordinates \hat{x}_{E}, ŷ_{E}, \hat{z}_{E}) sits at the center point e of the eye with axes parallel to {W} when the eye is unactuated by the robot.

The notations used are defined below.
i=1,2 refers to an index referring to one of the two arms.
{A} refers to an arbitrary right handed coordinate frame with {\hat{x}_{A}, ŷ_{A}, \hat{z}_{A}} as it is associated unit vectors and point a as the location of its origin.
V_{A/B}^{C},ω_{A/B}^{C} refers to the relative linear and angular velocities of frame {A} with respect to frame {B}, expressed in frame{C}. Unless specifically stated, all vectors are expressed in {W}.
v_{A}, ω_{A} refers to the absolute linear and angular velocities of frame {A}.
^{A}R_{B} refers to the rotation matrix of the moving frame {B} with respect to the frame {A}.
Rot(\hat{x}_{A}, α) refers to the rotation matrix about unit vector \hat{x}_{A} by an angle α.
[b×] refers to the skew symmetric cross product (i.e., a square matrix A such that it is equal to the negative of its transposed matrix, A=−A^{t}, where superscript t refers to the transpose operator) matrix of b.
\dot{q}_{P}_{ᵢ}=[\dot{q}_{P}_{ᵢ}₁, \dot{q}_{P}_{ᵢ}₂, \dot{q}_{P}_{ᵢ}₃, \dot{q}_{P}_{ᵢ}₄, \dot{q}_{P}_{ᵢ}₅, \dot{q}_{P}_{ᵢ}₆]^{t} refers to the joint speeds of the i^{th} parallel robot platform.
\dot{q}_{S}_{ᵢ}=[\dot{q}_{S}_{ᵢ}₁, \dot{q}_{S}_{ᵢ}₂]^{t} refers to the joint speeds of the serial robot. The first component can be the rotation speed about the axis of the serial robot support tube 505 and the second component can be the bending angular rate of the pre-bent cannula 520.
\dot{x}_{A}=[\dot{x}_{A}, \dot{y}_{A}, ż_{A}, ω_{Ax}, ω_{Ay}, ω_{Az}]^{t} refers to the twist of a general coordinate system {A}. For example, referring to FIG. 9A, {Qᵢ} represents the coordinate system defined by its three coordinate axes {\hat{x}_{Q}_{ᵢ}, ŷ_{Q}_{ᵢ}, \hat{z}_{Q}_{_{i}}}
\dot{x}_{P}_{ᵢ}=[\dot{x}_{P}_{ᵢ}, \dot{y}_{P}_{ᵢ}, ż_{P}_{ᵢ}, ω_{P}_{ᵢ}_{y}, ω_{P}_{ᵢ}_{Z}]^{t} refers to the twist of the moving platform of the i^{th} parallel robot where i=1,2.
\dot{x}ₑ refers to the twist of the i^{th} insertion needle end/base of the snake (e.g., the length of the NiTi cannula).
\dot{X}ₑ represents only the angular velocity of the eye (a 3×1 column vector). This is an exception to other notation because it is assumed that the translations of the center of motion of the eye are negligible due to anatomical constraints
^{A}\vec{ab} refers to the vector from point a to b expressed in frame {A}.
r refers to the bending radius of the pre-curved cannula.

$$W ( \vec{a} ) = [ \begin{matrix}{I}_{3 \times 3} & [ - ( \vec{a} ) \times ] \\ {0}_{3 \times 3} & {I}_{3 \times 3}\end{matrix} ]$$

refers to the twist transformation operator. This operator can be defined as a function of the translation of the origin of the coordinate system indicated by vector \vec{a}. W can be a 6×6 upper triangular matrix with the diagonal elements being a 3×3 unity matrix

$$[ \begin{matrix}100 \\ 010 \\ 001\end{matrix} ]$$

and the upper right 3×3 block being a cross product matrix and the lower left 3×3 block being all zeros.

In some embodiments, the kinematic modeling of the system includes the kinematic constraints due to the incision points in the eye and the limited degrees of freedom of the eye. Below, the kinematics of a two-armed robot with the eye are described, while describing the relative kinematics of a serial robot end effector with respect to a target point on the retina.

The Jacobian of the parallel robot platform, relating the twist of the moving platform frame {Pᵢ} to the joint speeds \dot{q}_{P}_{ᵢ} can be given by:

J_{P}_{ᵢ}\dot{x}_{P}_{ᵢ}=\dot{q}_{P}_{ᵢ} (1)

Developing the next step in the kinematic chain of the i^{th} hybrid robot, to {Qᵢ}, the linear and angular velocities can be expressed with respect to the respective velocities of the moving platform:

v_{Q}_{ᵢ}=v_{P}_{ᵢ}+ω_{P}_{ᵢ}×(\vec{pᵢqᵢ}) (2)

ω_{Q}_{ᵢ}=ω_{P}_{ᵢ} (3)

Writing equations (2) and (3) in matrix form results in the twist of the distal end of the adjustable lockable link:

\dot{x}_{Q}_{ᵢ}=Aᵢ\dot{x}_{P}_{ᵢ} (4)

where Aᵢ=W(\vec{pᵢqᵢ}) can be the twist transformation matrix.

The kinematic relationship of the frame {Nᵢ} can be similarly related to {Qᵢ} by combining the linear and angular velocities. The linear and angular velocities are:

v_{N}_{ᵢ}=v_{Q}_{ᵢ}+ω_{Q}_{ᵢ}×(\vec{qᵢnᵢ}) (5)

ω_{N}_{ᵢ}=ω_{Q}_{ᵢ}+\dot{q}_{S}_{ᵢ}₁\hat{z}_{Q}_{ᵢ} (6)

Equations 5 and 6 expressed in matrix form yield:

$${\dot{x}}_{{N}_{i}} = {B}_{i} {\dot{x}}_{{Q}_{i}} + [ \begin{matrix}0 \\ {\hat{z}}_{{Q}_{i}}\end{matrix} ] {\dot{q}}_{{s}_{i} 1} \qquad ( 7 )$$

where Bᵢ=W(\vec{qᵢnᵢ}).

Continuing to the final serial frame in the hybrid robot, {Gᵢ}, the linear and angular velocities can be written as

v_{G}_{ᵢ}=v_{N}_{ᵢ}+\dot{q}_{S}_{ᵢ}₂r\hat{z}_{G}_{ᵢ}+ω_{N}_{ᵢ}×(\vec{nᵢgᵢ}) (8)

ω_{G}_{ᵢ}=ω_{N}_{ᵢ}+\dot{q}_{S}_{ᵢ}₂ŷ_{N}_{ᵢ} (9)

Equations 8 and 9 expressed in matrix form yield:

$${\dot{x}}_{{G}_{i}} = {C}_{i} {\dot{x}}_{{N}_{i}} + [ \begin{matrix}r {\hat{z}}_{{G}_{i}} \\ {\hat{y}}_{{N}_{i}}\end{matrix} ] {\dot{q}}_{{s}_{i} 2} \qquad ( 10 )$$

where Cᵢ=W(\vec{nᵢgᵢ}).

To express the kinematics of the frame of the robot end effector, {Gᵢ}, as a function of the joint parameters of the i^{th} hybrid robotic system, the serial relationships developed above can be combined. Beginning with the relationship between the twist of frame {Gᵢ} and {Nᵢ} and inserting the relationship between {Nᵢ} and {Qᵢ} yields:

$${\dot{x}}_{{G}_{i}} = {C}_{i} {B}_{i} {\dot{x}}_{{Q}_{i}} + {C}_{i} [ \begin{matrix}0 \\ {\hat{z}}_{{Q}_{i}}\end{matrix} ] {\dot{q}}_{{s}_{i} 1} + [ \begin{matrix}r {\hat{z}}_{{G}_{i}} \\ {\hat{y}}_{{N}_{i}}\end{matrix} ] {\dot{q}}_{{s}_{i} 2} \qquad ( 11 )$$

Further, by reintroducing the matrix Cᵢ to the \dot{q}_{S,1} term, the serial joints of the hybrid system can be parameterized as follows:

\dot{x}_{G}_{ᵢ}=CᵢBᵢ\dot{x}_{Q}_{ᵢ}+J_{S}_{ₛ}\dot{q}_{S}_{ᵢ} (12)

where

$${J}_{{s}_{i}} = [ \begin{matrix}[ ( - {{n}_{i} {g}_{i}}^{} ) \times ] {\hat{z}}_{{Q}_{i}} & r {\hat{z}}_{{G}_{i}} \\ {\hat{z}}_{{Q}_{i}} & {\hat{y}}_{{N}_{i}}\end{matrix} ]$$

represents the Jacobian of the serial robot including the speeds of rotation about the axis of the serial robot cannula and the bending of the pre-curved cannula 520.

Inserting the relationship between {Qᵢ} and {pᵢ} and the inverse of the Stewart Jacobian equation (1), and condensing terms yields the final Jacobian for the i^{th} hybrid robot yields:

\dot{x}_{G}_{ᵢ}=Jₕ_{ᵢ}\dot{q}ₕ_{ᵢ} (13)

where Jₕ_{ᵢ}=[CᵢBᵢAᵢJ_{P}_{ᵢ}⁻¹,J_{S}_{_{i].}}

The eye can be modeled as a rigid body constrained to spherical motion by the geometry of the orbit and musculature. Roll-Pitch-Yaw angles (α,β,γ) can be chosen to describe the orientation of the eye such that the rotation matrix ^{w}Rₑ specifies the eye frame {E} with respect to {W} as ^{w}Rₑ=R_{z}R_{y}Rₓ where Rₓ=Rot(\hat{x}_{W},α), R_{y}=Rot(ŷ_{W},β), and R_{z}=Rot(\hat{z}_{W},γ).

The angular velocity of the eye can be parameterized by:

\dot{x}ₑ=[\dot{α},\dot{β},\dot{γ}]^{t} (14)

The kinematics of the end effector with respect to the eye can also be modeled. For example, with the kinematics of the eye and the i^{th} hybrid robotic system characterized separately, the formulations can be combined to define the kinematic structure of the eye and i^{th} hybrid robot. This relationship can allow expression of the robot joint parameters based on the desired velocity of the end effector with respect to the eye and the desired angular velocity of the eye. To achieve this relationship, an arbitrary goal point on the retinal surface tᵢ can be chosen. The angular velocity of the eye imparts a velocity at point tᵢ

vₜ_{ᵢ}=Tᵢ\dot{x}ₑ (15)

where end effector Tᵢ=└(−\vec{etᵢ})x┘

The linear velocity of the end effector frame of the robot with respect to the goal point tᵢ can be written as:

v_{g}_{ᵢ}_{/t}_{ᵢ}=v_{g}_{ᵢ}−vₜ_{ᵢ} (16)

Inserting equations (13) and equations (15) into equation (16) yields a linear velocity of the end effector as a function of the robot joint speeds and the desired eye velocity

v_{g}_{ᵢ}_{/t}_{ᵢ}=[I_{3×3},0_{3×3}]Jₕ\dot{q}ₕ_{ᵢ}−Tᵢ\dot{x}ₑ (17)

Similarly, the angular velocity of the end effector frame of the robot with respect to the eye frame can be written as:

ω_{g}_{ᵢ}_{/e}=ω_{g}_{ᵢ}−ωₑ (18)

or, by inserting equation (13) and equation (15) into equation (18) yielding

ω_{g}_{ᵢ}_{/e}=[0_{3×3},I_{3×3}]Jₕ_{ᵢ}\dot{q}ₕ_{ᵢ}−\dot{x}ₑ (19)

further combining the linear equation (17) and angular equation (19) velocities yields the twist of the end effector with respect to point tᵢ:

\dot{x}_{g}_{ᵢ}_{/t}_{ᵢ}=Jₕ_{ᵢ}\dot{q}ₕ_{ᵢ}−Dᵢ\dot{x}ₑ (20)

where Dᵢ=[Tᵢ^{t},I_{3×3}]^{t}.

In some embodiments, the mechanical structure of the hybrid robot in the eye (e.g., vitreous cavity) allows only five degrees of freedom as independent rotation about the \hat{z}_{G}_{ᵢ} axis can be unachievable. This rotation can be easily represented by the third w-v-w Euler angle φᵢ. It should be noted that the first angle φᵢ represents the rotation between the projection of the \hat{z}_{G}_{ᵢ} axis on the \hat{x}_{W}ŷ_{W} plane and \hat{x}_{W} and the second angle θᵢ represents rotation between \hat{z}_{W} and \hat{z}_{G}_{ᵢ}

The system can utilize path planning and path control. For example, path planning and path control can be used to ease the surgery by having the tele-robotic master controller automatically perform some of the movements for the slave hybrid-robot. For the purposes of path planning and control, the twist of the system can therefore be parameterized with w-v-w Euler angles and the third Euler angle eliminated by a degenerate matrix Kᵢ defined as follows:

{dot over (\tilde{x}_{g}_{ᵢ}_{/t}_{ᵢ}=Kᵢ\dot{x}_{g}_{ᵢ}_{/t}_{ᵢ} (21)

Inserting this new parameterization into the end effector twist yields a relation between the achievable independent velocities and the joint parameters of the hybrid system.

{dot over (\tilde{x}_{g}_{ᵢ}ₜ_{ᵢ}+KᵢDᵢ\dot{x}ₑ=KᵢJₕ_{ᵢ}\dot{q}ₕ_{ᵢ} (22)

The robotic system can be constrained such that the hybrid robots move in concert (e.g., move substantially together) to control the eye without injuring the structure by tearing the insertion points. This motion can be achieved by allowing each insertion arm to move at the insertion point only with the velocity equal to the eye surface at that point, plus any velocity along the insertion needle (which can be support tube 505, pre-bent tube 520 or guide wire 635). This combined motion constrains the insertion needle to the insertion point without damage to the structure.

To assist in the development of the aforementioned constraint, point mᵢ can be defined at the insertion point on the sclera surface of the eye and m; can be defined as point on the insertion needle instantaneously coincident with mᵢ. To meet the above constraint, the velocity of m′ᵢ must be equal to the velocity of point mᵢ in the plane perpendicular to the needle axis:

v_{m′}_{ᵢ}_{⊥}=vₘ_{ᵢ}_{⊥} (23)

Taking a dot product in the directions, \hat{X}_{Q}_{ᵢ} and ŷ_{Q}_{ᵢ} yields two independent constraint equations:

\hat{x}_{Q}_{ᵢ}^{t}v_{m′}_{ᵢ}=\hat{x}_{Q}_{ᵢ}^{t}vₘ_{ᵢ} (24)

ŷ_{Q}_{ᵢ}^{t}v_{m′}_{ᵢ}=ŷ_{Q}_{ᵢ}^{t}vₘ_{ᵢ} (25)

These constraints can be expressed in terms of the joint angles by relating the velocities of point mᵢ and m′ᵢ to the robot coordinate systems. The velocity of point m; can be related to the velocity of frame {Qᵢ} as follows:

v_{m′}_{ᵢ}=v_{Q}_{ᵢ}+ω_{Q}_{ᵢ}×\vec{qᵢmᵢ} (26)

By substituting the twist of frame {Qᵢ}, the above equation becomes:

v_{m′}_{ᵢ}=[I_{3×3},0_{3×3}]\dot{x}_{Q}_{ᵢ}+Eᵢ[0_{3×3},I_{3×3}]\dot{x}_{Q}_{ᵢ} (27)

where Eᵢ=[\vec{qᵢmᵢ}×].

Inserting equations (4) and (1) and writing in terms of the hybrid joint parameters \dot{q}ₕ_{ᵢ} yields:

v_{m′}_{ᵢ}=Fᵢ\dot{q}ₕ_{ᵢ} (28)

where Fᵢ=([I_{3×3},0_{3×3}]−Eᵢ[0_{3×3},I_{3×3}])AᵢJ_{P}_{ᵢ}⁻¹[I_{6×6},0_{6×2].}

An expression for the velocity of the insertion point mᵢ can be related to the desired eye velocity, similar to the derivation of velocity of point tᵢ, yielding:

vₘ_{ᵢ}=Mᵢ\dot{x}ₑ (29)

where Mᵢ=└(−\vec{emᵢ})x┘.

Substituting equation (28) and equation (29) into equation (24) and equation (25) yields the final constraint equations given for the rigid body motion of the eye-robot system:

\hat{x}_{Q}_{ᵢ}^{t}Fᵢ\dot{q}ₕ_{ᵢ}=\hat{x}_{Q}_{ᵢ}^{t}Mᵢ\dot{x}ₑ (30)

ŷ_{Q}_{ᵢ}^{t}Fᵢ\dot{q}ₕ_{ᵢ}Mᵢ\dot{x}ₑ (31)

Combining these constraints with the twist of the hybrid systems for indices 1 and 2, yields the desired expression of the overall eye-robotic system relating the hybrid robotic joint parameters to the desired end effector twists and the desired eye velocity.

$$[ \begin{matrix}{K}_{1} {J}_{{h}_{1}} & {0}_{5 \times 8} \\ {0}_{5 \times 8} & {K}_{2} {J}_{{h}_{2}} \\ {G}_{1} {F}_{1} & {0}_{2 \times 8} \\ {0}_{2 \times 8} & {G}_{2} {F}_{2}\end{matrix} ] [ \begin{matrix}{\dot{q}}_{{h}_{1}} \\ {\dot{q}}_{{h}_{2}}\end{matrix} ] = [ \begin{matrix}{I}_{5 \times 5} & {0}_{5 \times 5} & {K}_{1} {D}_{1} \\ {0}_{5 \times 5} & {I}_{5 \times 5} & {K}_{2} {D}_{2} \\ {0}_{2 \times 5} & {0}_{2 \times 5} & {G}_{1} {M}_{1} \\ {0}_{2 \times 5} & {0}_{2 \times 5} & {G}_{2} {M}_{2}\end{matrix} ] [ \begin{matrix}{{x}^{\tilde{.}}}_{{g}_{1} / {t}_{1}} \\ {{x}^{\tilde{.}}}_{{g}_{2} / {t}_{2}} \\ {\dot{x}}_{e}\end{matrix} ] \qquad ( 32 )$$

where Gᵢ=[\hat{x}_{Q}_{ᵢ},ŷ_{Q}_{_{i]}}^{t}.

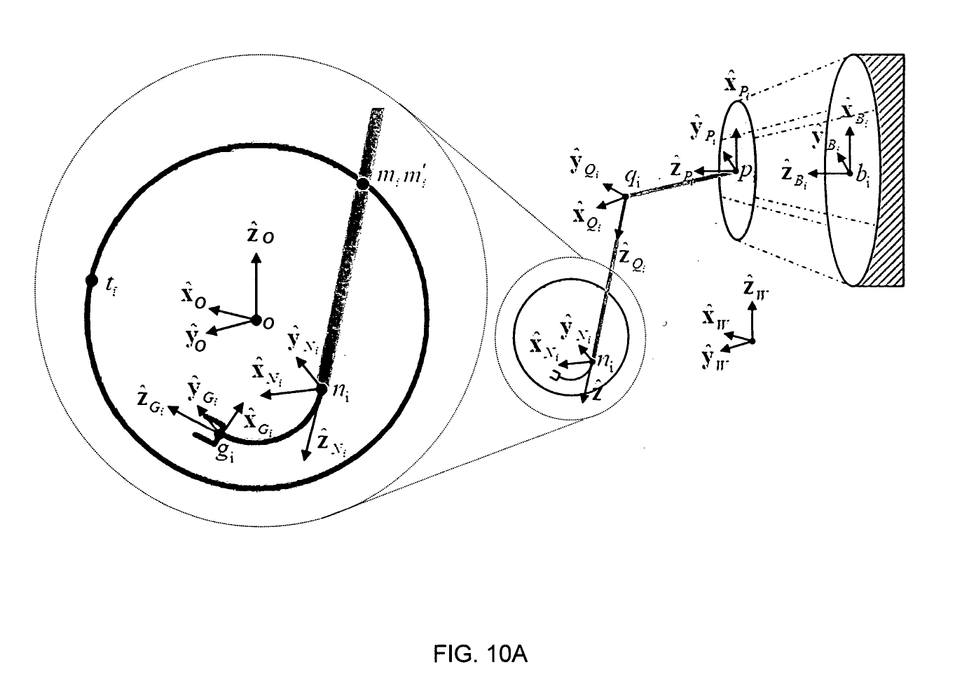

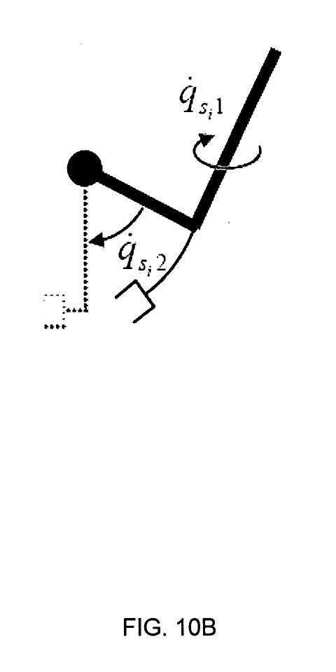

Referring to FIG. 10A-10B, an organ and the i^{th} hybrid robotic arm is displayed. The organ is enlarged (FIG. 10A) for a clearer view of the end effector and the organ coordinate frames. FIG. 10B illustratively displays an enlarged view of the end effector. The following coordinate systems are defined to assist in the derivation of the system kinematics. The world coordinate system {W} (having coordinates \hat{x}_{W}, ŷ_{W}, \hat{z}_{W}) can be centered at an arbitrarily predetermined point in the patient's forehead with the patient in a supine position. The \hat{z}_{W} axis points vertically and ŷ_{W} axis points superiorly. The parallel robot base coordinate system {Bᵢ} (having coordinates \hat{x}_{B}_{ᵢ}, ŷ_{B}_{ᵢ}, \hat{z}_{B}_{ᵢ}) of the i^{th} hybrid robot can be located at point bᵢ (i.e., the center of the base platform) such that the \hat{z}_{B}_{ᵢ} axis lies perpendicular to the base of the parallel robot platform and the \hat{X}_{B}_{ᵢ} axis lies parallel to \hat{z}_{W}. The moving platform coordinate system of the i^{th} hybrid robot {Pᵢ} (having coordinates \hat{x}ₚ_{ᵢ}, ŷ_{P}_{ᵢ}, \hat{z}_{P}_{ᵢ}) lies in center of the moving platform, at point pᵢ such that the axes lie parallel to {Bᵢ} when the parallel robot platform lies in the home configuration (e.g., the initial setup position). The parallel robot extension arm coordinate system of the i^{th} hybrid {Qᵢ} (having coordinates \hat{x}_{Q}_{ᵢ}, ŷ_{Q}_{ᵢ}, \hat{z}_{Q}_{ᵢ}) can be attached to the distal end of the arm at point qᵢ, with \hat{z}_{Q}_{ᵢ} lying along the direction of the insertion needle of the robot \vec{qᵢnᵢ}, and \hat{x}_{Q}_{ᵢ} fixed during setup procedure. The serial robot (e.g., intra-ocular dexterity robot) base coordinate system of the i^{th} hybrid robot {Nᵢ} (having coordinates \hat{x}_{N}_{ᵢ} ŷ_{N}_{ᵢ} \hat{z}_{N}_{ᵢ}) lies at point nᵢ with the \hat{z}_{N}_{ᵢ} axis also pointing along the insertion needle length \vec{qᵢnᵢ} and the ŷ_{N}_{ᵢ} axis rotated from ŷ_{Q}_{ᵢ} an angle q_{S}_{ᵢ}₁ about \hat{z}_{N}_{ᵢ}. The end effector coordinate system {Gᵢ} (having coordinates \hat{x}_{G}_{ᵢ}, ŷ_{G}_{ᵢ}, \hat{z}_{Q}_{ᵢ}) lies at point gᵢ with the \hat{z}_{G}_{ᵢ}, axis pointing in the direction of the end effector gripper and the ŷ_{G}_{ᵢ} axis parallel to the ŷ_{N}_{ᵢ}, axis. The organ coordinate system {O} (having coordinates \hat{x}_{O}, ŷ_{O}, \hat{z}_{O})sits at the rotating center o of the organ with axes parallel to {W} when the organ can be not actuated by the robot.

The additional notations used are defined below:
i refers to the index identifying each robotic arm. Further, for unconstrained organs i=1, 2, 3 while for the eye i=1,2.
{A} refers to a right handed coordinate frame with, \hat{x}_{A}, ŷ_{A}, \hat{z}_{A} as its associated unit vectors and point a as the location of its origin.
V_{A/B}^{C},ω_{A/B}^{C} refers to the relative linear and angular velocities of frame {A} with respect to {B}, expressed in {C}. It will be understood that, unless specifically stated, all vectors displayed below can be expressed in {W}.
v_{A},ω_{A} refers to absolute linear and angular velocities of frame {A}.
^{A}R_{B} refers to the rotation matrix of the moving frame {B} with respect to {A}.
Rot(\hat{x}_{A},α) refers to the rotation matrix about unit vector by angle α.
[b×] refers to the skew symmetric cross product matrix of vector b.
\dot{q}_{P}_{ᵢ}=[\dot{q}_{P}_{ᵢ}₁, \dot{q}_{P}_{ᵢ}₂, \dot{q}_{P}_{ᵢ}₃, \dot{q}_{P}_{ᵢ}₄, \dot{q}_{P}_{ᵢ}₅, \dot{q}_{P}_{ᵢ}₆]^{t} refers to the active joint speeds of the i^{th} parallel robot platform.
\dot{q}_{S}_{ᵢ}=[\dot{q}_{S}_{ᵢ}₁,\dot{q}_{S}_{ᵢ}₂]^{t} refers to the joint speeds of the i^{th} serial robot (e.g., intra-ocular dexterity robot). The first component can be the rotation speed about the axis of the serial robot (e.g., intra-ocular dexterity robot) tube, and the second component can be the bending angular rate of the pre-bent tube 520.
\dot{x}_{A}, \dot{x}_{P}_{ᵢ}, \dot{x}_{O} refers to the twists of frame {A}, of the i^{th} parallel robot moving platform, and of the organ.
^{A}\vec{ab} refers to the vector from point a to b expressed in frame {A}.
L_{S} refers to the bending radius of the pre-bent tube 520 of the serial robot (e.g., intra-ocular dexterity robot).

$$W ( {a}^{->} ) = [ \begin{matrix}{I}_{3 \times 3} & [ - ( {a}^{->} ) \times ] \\ {0}_{3 \times 3} & {I}_{3 \times 3}\end{matrix} ]$$

refers to the twist transformation operator. This operator can be defined as a function of the translation of the origin of the coordinate system indicated by vector \vec{a} can be a 6×6 upper triangular matrix with the diagonal elements being a 3×3 unity matrix

$$[ \begin{matrix}100 \\ 010 \\ 001\end{matrix} ]$$

and the upper right 3×3 block being a cross product matrix and the lower left 3×3 block being all zeros.

In some embodiments, the kinematic modeling of the system can include the kinematic constraints of the incision points on the hollow organ. Below, the kinematics of the triple-armed robot with the organ and describes the relative kinematics of the serial robot (e.g., intra-ocular dexterity robot) end effector with respect to a target point on the organ.

The Jacobian of the parallel robot platform relating the twist of the moving platform frame \dot{x}_{P}_{ᵢ} to the joint parameters, \dot{q}_{P}_{ᵢ} is shown in equation 33. Further, the overall hybrid Jacobian matrix for one robotic arm is obtained as equation 34.

J_{P}_{ᵢ}\dot{x}_{P}_{ᵢ}=\dot{q}_{P}_{ᵢ} (33)

\dot{x}_{G}_{ᵢ}=Jₕ_{ᵢ}\dot{q}ₕ_{ᵢ} (34)

In some embodiments, modeling can be accomplished by considering the elasticity and surrounding anatomy of the organ. Further, in some embodiments, the below analysis does not include the organ elasticity. Further still, a six dimension twist vector can be used to describe the motion of the organ using the following parameterization:

\dot{x}ₒ=[\dot{x}ₒₗ^{t},\dot{x}ₒₐ^{t}]^{t}=[\dot{x},\dot{y},ż,\dot{α},\dot{β},\dot{γ}]^{t} (35)

where x, y, z, α, β, γ can be linear positions and Roll-Pitch-Yaw angles of the organ, and \dot{x}ₒₗ and \dot{x}ₒₐ correspond to the linear and angular velocities of the organ respectively.

In some embodiments, the Kinematics of the serial robot (e.g., intra-ocular dexterity robot) end effector with respect to the organ can be modeled. Further, in some embodiments, the model can express the desired velocity of the end effector with respect to the organ and the desired velocity of the organ itself, an arbitrary target point tᵢ on the inner surface of the organ can be chosen. The linear and angular velocities of the end effector frame with respect to the target point can be written as:

v_{g}_{ᵢ}_{/t}_{ᵢ}=[I_{3×3},0_{3×3}]Jₕ_{ᵢ}\dot{q}ₕ_{ᵢ}−\dot{x}ₒₗ−Tᵢ\dot{x}ₒₐ (36)

ω_{g}_{ᵢ}_{/o}=[0_{3×3},I_{3×3}]Jₕ_{ᵢ}\dot{q}ₕ_{ᵢ}−\dot{x}ₒₐ (37)

Further, combining equation 36 and equation 37 yields the twist of the end effector with respect to point tᵢ:

\dot{x}_{g}_{ᵢ}_{/t}_{ᵢ}=Jₕ_{ᵢ}\dot{q}ₕ_{ᵢ}−Hᵢ\dot{x}ₒ (38)

where Tᵢ=└(−\vec{otᵢ})×┘ and

$${H}_{i} = [ \begin{matrix}{I}_{3 \times 3} & {T}_{i} \\ {0}_{3 \times 3} & {I}_{3 \times 3}\end{matrix} ]$$

The mechanical structure of the hybrid robot in the organ cavity can allow only five degrees of freedom as independent rotation of the serial robot (e.g., intra-ocular dexterity robot) end effector about the \hat{z}_{G}_{ᵢ} axis can be unachievable due to the two degrees of freedom of the serial robot (e.g., intra-ocular dexterity robot). This rotation can be represented by the third w-v-w Euler angle φᵢ. In some embodiments, for the purposes of path planning and control, the twist of the system can be parameterized using w-v-w Euler angles while eliminating the third Euler angle through the use of a degenerate matrix Kᵢ as defined below. Inserting the aforementioned parameterization into the end effector twist, equation 38, yields a relation between the achievable independent velocities and the joint parameters of the hybrid system, equation 40.

{dot over (\tilde{x}_{g}_{ᵢ}_{/t}_{ᵢ}=Kᵢ\dot{x}_{g}_{ᵢ}_{/t}_{ᵢ} (39)

{dot over (\tilde{x}_{g}_{ᵢ}_{/t}_{ᵢ}+KᵢHᵢ\dot{x}ₒ=KᵢJₕ_{ᵢ}\dot{q}ₕ_{ᵢ} (40)

In some embodiments, the robotic system can be constrained such that the hybrid arms move synchronously to control the organ without tearing the insertion point. For example, the robotic system can be constrained such that the multitude, nₐ, of hybrid robotic arms moves synchronously to control the organ without tearing the insertion points. The i^{th} incision point on the organ be designated by point mᵢ, i=1,2,3 . . . nₐ. The corresponding point, which can be on the serial robot (e.g., intra-ocular dexterity robot) cannula of the i^{th} robotic arm and instantaneously coincident with mᵢ, be designated by m′ᵢ, i=1,2,3 . . . nₐ. In some embodiments, to prevent damage to the anatomy, an equality constraint must be imposed between the projections of the linear velocities of mᵢ and m′ᵢ on a plane perpendicular to the longitudinal axis of the i^{th} serial robot (e.g., intra-ocular dexterity robot) cannula. These conditions can be given in equation 41 and equation 42 as derived in detail below.

\hat{x}_{Q}_{ᵢ}^{t}Fᵢ\dot{q}ₕ_{ᵢ}=\hat{x}_{Q}_{ᵢ}^{t}(\dot{x}ₒₗ+Mᵢ\dot{x}ₒₐ),i=1,2,3 . . . nₐ (41)

\hat{x}_{Q}_{ᵢ}^{t}Fᵢ\dot{q}ₕ_{ᵢ}=ŷ_{Q}_{ᵢ}^{t}(\dot{x}ₒₗ+Mᵢ\dot{x}ₒₐ),i=1,2,3 . . . nₐ (42)

Equation 41 and equation 42 can constitute 2nₐ scalar equations that provide the conditions for the organ to be constrained by nₐ robotic arms inserted into it through incision points. For the organ to be fully constrained by the robotic arms, equation 41 and equation 42 should have the same rank as the dimension of the organ twist, \dot{x}ₒ as constrained by its surrounding anatomy. Further, if the organ is a free-floating organ, then the rank should be six and therefore a minimum of three robotic arms can be necessary to effectively stabilize the organ. Further still, if the organ is constrained from translation (e.g., as for the eye), the required rank can be three and hence the minimum number of arms can be two (e.g., for a dual-arm ophthalmic surgical system).

Combining the constraint equations as derived below with the twist of the hybrid robotic arms {dot over (\tilde{x}_{g}_{ᵢ}_{/t}_{ᵢ} for i=1, 2, 3, yields the desired expression of the overall organ-robotic system relating the joint parameters of each hybrid robotic arm to the desired end effector twists and to the organ twist.

$$[ \underbrace{\begin{matrix}{K}_{1} {J}_{{h}_{1}} & {0}_{5 \times 8} & {0}_{5 \times 8} \\ {0}_{5 \times 8} & {K}_{2} {J}_{{h}_{2}} & {0}_{5 \times 8} \\ {0}_{5 \times 8} & {0}_{5 \times 8} & {K}_{3} {J}_{{h}_{3}} \\ {G}_{1} {F}_{1} & {0}_{2 \times 8} & {0}_{2 \times 8} \\ {0}_{2 \times 8} & {G}_{2} {F}_{2} & {0}_{2 \times 8} \\ {0}_{2 \times 8} & {0}_{2 \times 8} & {G}_{3} {F}_{3}\end{matrix}}_{{J}_{I}} ] [ \begin{matrix}{\dot{q}}_{{h}_{1}} \\ {\dot{q}}_{{h}_{2}} \\ {\dot{q}}_{{h}_{3}}\end{matrix} ] = [ \underbrace{\begin{matrix}{I}_{5 \times 5} & {0}_{5 \times 5} & {0}_{5 \times 5} & {K}_{1} {H}_{1} \\ {0}_{5 \times 5} & {I}_{5 \times 5} & {0}_{5 \times 5} & {K}_{2} {H}_{2} \\ {0}_{5 \times 5} & {0}_{5 \times 5} & {I}_{5 \times 5} & {K}_{3} {H}_{3} \\ {0}_{2 \times 5} & {0}_{2 \times 5} & {0}_{2 \times 5} & {G}_{1} {P}_{1} \\ {0}_{2 \times 5} & {0}_{2 \times 5} & {0}_{2 \times 5} & {G}_{2} {P}_{2} \\ {0}_{2 \times 5} & {0}_{2 \times 5} & {0}_{2 \times 5} & {G}_{3} {P}_{3}\end{matrix}}_{{J}_{O}} ] [ \begin{matrix}{{x}^{\tilde{.}}}_{{g}_{1} / {t}_{1}} \\ {{x}^{\tilde{.}}}_{{g}_{2} / {t}_{2}} \\ {{x}^{\tilde{.}}}_{{g}_{3} / {t}_{3}} \\ {\dot{x}}_{o}\end{matrix} ] \qquad ( 43 )$$

Considering the contact between fingers (e.g., graspers delivered into an organ) and the payload (e.g., the organ) a differential kinematic relationship can be modeled. Further, multi-arm manipulation can be modeled wherein the relative position between the robotic arms and the organ can be always changing. Further, by separating input joint rates \dot{q}ₕ output organ motion rates \dot{x}ₒ and relative motion rates {dot over (\tilde{x}_{g/t} equation 43, the kinematic relationship can be modeled.

The robot kinetostatic performance can be evaluated by examining the characteristics of the robot Jacobian matrix. Further, normalization of the Jacobian can be necessary when calculating the singular values of the Jacobian. These singular values can depend on the units of the individual cells of the Jacobian. Inhomogeneity of the units of the Jacobian can stem from the inhomogeneity of the units of its end effector twist and inhomogeneity of the units in joint space (e.g., in cases where not all the joints are of the same type, such as linear or angular). Normalizing the Jacobian matrix requires scaling matrices corresponding to ranges of joint and task-space variables by multiplying the Jacobian for normalization. Further, using the characteristic length to normalize the portion of the Jacobian bearing the unit of length and using a kinematic conditioning index defined as the ratio of the smallest and largest singular value of a normalized Jacobian the performance can be evaluated. Further still, the Jacobian scaling matrix can be found by using a physically meaningful transformation of the end effector twist that would homogenize the units of the transformed twist. The designer can be required to determine the scaling/normalization factors of the Jacobian prior to the calculation of the condition index of the Jacobian. The methodology used relies on the use of individual characteristic lengths for the serial and the parallel portions of each robotic arm.

Equations 44-46 specify the units of the individual vectors and submatrices of equation 43. The brackets can be used to designate units of a vector or a matrix, where [m] and [s] denote meters and seconds respectively. The Jacobian matrices J_{I} and Jₒ do not possess uniform units, and using a single characteristic length to normalize both of them may not be possible because the robotic arms can include both serial and parallel portions. Also, evaluating the performance of the robotic system for different applications can include simultaneously normalizing J_{I} and Jₒ rendering the units of all their elements to be unity. Further, this can be achieved through an inspection of the units of these matrices and the physical meaning of each submatrix in equation 43 while relating each matrix block to the kinematics of the parallel robot, or the serial robot (e.g., intra-ocular dexterity robot), or the organ.

$$\begin{matrix}[ {{x}^{\tilde{.}}}_{{g}_{i} / {t}_{i}} ] = {[ {[ m / s ]}_{1 \times 3} , {[ 1 / s ]}_{1 \times 2} ]}^{t} , \\ [ {\dot{x}}_{o} ] = {[ {{[ m / s ]}_{1 \times 3} [ 1 / s ]}_{1 \times 3} ]}^{t} \\ [ {\dot{q}}_{{h}_{i}} ] = {[ {[ m / s ]}_{1 \times 6} , {[ 1 / s ]}_{1 \times 2} ]}^{t} & ( 44 ) \\ [ {G}_{i} {P}_{i} ] = [ {{[ 1 ]}_{2 \times 3} [ m ]}_{2 \times 3} ] , [ {G}_{i} {F}_{i} ] = [ {{[ 1 ]}_{2 \times 6} [ 0 ]}_{2 \times 2} ] & ( 45 ) \\ [ {K}_{i} {H}_{i} ] = [ \begin{matrix}{[ 1 ]}_{3 \times 3} & {[ m ]}_{3 \times 3} \\ {[ 0 ]}_{2 \times 3} & {[ 1 ]}_{2 \times 3}\end{matrix} ] , \\ [ {K}_{i} {J}_{{h}_{i}} ] = [ \begin{matrix}{[ 1 ]}_{3 \times 6} & {[ m ]}_{3 \times 2} \\ {[ 1 / m ]}_{2 \times 6} & {[ 1 ]}_{2 \times 2}\end{matrix} ] & ( 46 )\end{matrix}$$

When the Jacobian matrix J_{O} characterizes the velocities of the rotating organ and the end effector, the matrix can be homogenized using the radius of the organ at the target point as the characteristic length. It can be this radius, as measured with respect to the instantaneous center of rotation that imparts a linear velocity to point tᵢ, as a result of the angular velocity of the organ. The top right nine components of J_{O} given by KᵢHᵢ i=1,2,3 of equation 43, bear the unit of [m]. Hence, dividing them by the radius of the organ at the target point, Lᵣ can render their units to be unity. The same treatment can be also carried out to the rightmost six components of each matrix block GᵢPᵢ i=1,2,3, where we divide them by Lᵣ as well.

The Jacobian matrix J_{I} can describe the geometry of both the parallel robot and the serial robot. Further this can be done by using both Lₚ, the length of the connection link of the parallel robot, \vec{pᵢqᵢ}, and L_{S} the bending radius of the inner bending tube of the serial robot, as characteristic lengths. In some instances, Lₚ is multiplied by those components in KᵢJₕ_{ᵢ} bearing the unit of [1/m]. Further, the components in KᵢJₕ_{ᵢ} that bear the unit of [m] can be divided by Lₛ. This can result in a normalized input Jacobian J_{I} that can be dimensionless. Further still, the radius of the moving platform can be used for normalization. Lₚ can be the scaling factor of the linear velocity at point qᵢ stemming from a unit angular velocity of the moving platform. Similarly, the circular bending cannula of the serial robot can be modeled as a virtual rotary joint, and the bending radius Lₛ can be used to normalize the components of KᵢJₕ_{ᵢ} that are related to the serial robot.

In some embodiments, the eye can be modeled as a constrained organ allowing only rotational motions about its center. This can be used to produce a simplified model of the twist of the organ as a three dimensional vector as indicated in equation 47. The relative linear and angular velocities of the robot arm end effector are given by equation 48 and equation 49 with respect to a target point t; on the retina. Equation 48 and equation 49 can be combined to yield the relative twist between the end effector of each arm and the target point, equation 50, where Dᵢ=[Tᵢ^{t},I_{3×3}]^{t}. Additionally, the five dimensional constrained twist of the serial robot end effector in equation 40 simplifies to equation 51. Further, the overall Jacobian equation for the whole system with the eye simplifies to equation 52.

$$\begin{matrix}{\dot{x}}_{e} = {[ \dot{α} , \dot{β} , \dot{γ} ]}^{t} & ( 47 ) \\ {v}_{{g}_{i} / {t}_{i}} = [ {I}_{3 \times 3} , {0}_{3 \times 3} ] {J}_{{h}_{i}} {\dot{q}}_{{h}_{i}} - {T}_{i} {\dot{x}}_{e} & ( 48 ) \\ {ω}_{{g}_{i} / e} = [ {0}_{3 \times 3} , {I}_{3 \times 3} ] {J}_{{h}_{i}} {\dot{q}}_{{h}_{i}} - {\dot{x}}_{e} & ( 49 ) \\ {\dot{x}}_{{g}_{i} / {t}_{i}} = {J}_{{h}_{i}} {\dot{q}}_{{h}_{i}} - {D}_{i} {\dot{x}}_{e} & ( 50 ) \\ {{x}^{\tilde{.}}}_{{g}_{i} / {t}_{i}} + {K}_{i} {D}_{i} {\dot{x}}_{e} = {K}_{i} {J}_{{h}_{i}} {\dot{q}}_{{h}_{i}} & ( 51 ) \\ \underbrace{[ \begin{matrix}{K}_{1} {J}_{{h}_{1}} & {0}_{5 \times 8} \\ {0}_{5 \times 8} & {K}_{2} {J}_{{h}_{2}} \\ {G}_{1} {F}_{1} & {0}_{2 \times 8} \\ {0}_{2 \times 8} & {G}_{2} {F}_{2}\end{matrix} ]}_{M} [ \begin{matrix}{\dot{q}}_{{h}_{1}} \\ {\dot{q}}_{{h}_{2}}\end{matrix} ] = \underbrace{\begin{matrix}[ \underbrace{\begin{matrix}{I}_{5 \times 5} & {0}_{5 \times 5} \\ {0}_{5 \times 5} & {I}_{5 \times 5} \\ {0}_{2 \times 5} & {0}_{2 \times 5} \\ {0}_{2 \times 5} & {0}_{2 \times 5}\end{matrix}}_{{N}_{1}} & \underbrace{\begin{matrix}{K}_{1} {D}_{1} \\ {K}_{2} {D}_{2} \\ {G}_{1} {M}_{1} \\ {G}_{2} {M}_{2}\end{matrix}}_{{N}_{2}} ]\end{matrix}}_{N} [ \begin{matrix}{{x}^{\tilde{.}}}_{{g}_{1} / {t}_{1}} \\ {{x}^{\tilde{.}}}_{{g}_{2} / {t}_{2}} \\ {\dot{x}}_{e}\end{matrix} ] & ( 52 )\end{matrix}$$

In some embodiments, at least four modes of operation can be performed by a robotic system for surgery: intra-organ manipulation and stabilization of the organ; organ manipulation with constrained intra-organ motions (e.g., manipulation of the eye while maintaining the relative position of devices in the eye with respect to a target point inside the eye); organ manipulation with unconstrained intra-organ motion (e.g., eye manipulation regardless of the relative motions between devices in the eye and the eye); and simultaneous organ manipulation and intra-organ operation.

Further, each of the aforementioned four modes can be used to provide a dexterity evaluation. For example, intra-organ operation with organ stabilization can be used to examine the intraocular dexterity, a measure of how well this system can perform a specified surgical task inside the eye with one of its two arms. Further, for example, organ manipulation with constrained intra-organ motions can be used to evaluate orbital dexterity, a measure of how well the two arms can grossly manipulate the rotational position of eye, while respecting the kinematic constraints at the incision points and maintaining zero velocity of the grippers with respect to the retina. Still further, for example, organ manipulation with unconstrained intra-organ motion, can be used to evaluate the orbital dexterity without constraints of zero velocity of the grippers with respect to the retina. Still further, for example, simultaneous organ manipulation and intra-organ operation can be used to measure of intra-ocular and orbital dexterity while simultaneously rotating the eye and executing an intra-ocular surgical task.

It will be understood that for the analysis below both robotic arms are put to the side of the eyeball. Two incision points can be specified by angles [π/3,π/3]^{t} and [π/3,π]^{t}. The aforementioned four modes of surgical tasks can all be based on this setup.

Rewriting equation 52 using matrices M and N, equation 53 can be obtained where \dot{q}ₕ=[\dot{q}ₕ_{₁}^{t},\dot{q}ₕ_{₂}^{t}]^{t} and {dot over (\tilde{x}_{g/t}=[{dot over (\tilde{x}_{g}_{₁}_{/t}_{₁}^{t}, {dot over (\tilde{x}_{g}_{₂}_{/t}_{₂}^{t}]^{t}. Specifying \dot{x}ₑ=0 equation 53 simplifies to equation 54 and its physical meaning can be that the angular velocity of the eye is zero. Equation 54 represents the mathematical model of intra-ocular manipulation while constraining the eye.

Similarly, specifying {dot over (\tilde{x}_{g/t}=0 equation 53 can simplify to equation 55. Physically this signifies that by specifying the relative velocities of the serial robot end effector with respect to the eye to be zero, equation 55 represents the mathematical model of orbital manipulation.

M\dot{q}ₕ=N₁{dot over (\tilde{x}_{g/t}+N₂\dot{x}ₑ (53)

M\dot{q}ₕ=Nᵢ{dot over (\tilde{x}_{g/t} (54)

M\dot{q}ₕ=N₂\dot{x}ₑ (55)

For intra-organ operation with organ stabilization, two modular configurations can be taken into account. In the first configuration the robotic arms can use standard ophthalmic instruments with no distal dexterity (e.g., a straight cannula capable of rotating about its own longitudinal axis). This yields a seven degree of freedom robotic arm. The Jacobian matrix for a seven degree of freedom robotic arm can be

$${J}_{{7}_{i}} = [ {B}_{i} {A}_{i} {J}_{{P}_{i}}^{- 1} , \begin{matrix}{0}_{3 \times 1} \\ {\hat{z}}_{{Q}_{i}}\end{matrix} ]$$

as in equation 56 and equation 57. In the second configuration the robotic arms employ the serial robot, therefore a kinematic model can be represented by equation 34. An intra-ocular dexterity evaluation can be used to compare the performance of the system in both these configurations (e.g., with or without the serial robot).

The method of using multiple characteristic lengths to normalize the overall Jacobian can be used for the purpose of performance evaluation. For intra-organ operation with organ stabilization, evaluating translational and rotational dexterity separately can be accomplished by investigating the upper and lower three rows of J₇_{ᵢ} and Jₕ_{ᵢ}. Equation 56 and equation 58 can give the normalized sub-Jacobians for translational motions of seven degree of freedom and eight degree of freedom robots, while equation 57 and equation 59 can give the normalized sub-Jacobians for rotational motions of seven degree of freedom and eight degree of freedom robots.

$$\begin{matrix}{J}_{7 DoF\_t} = [ {I}_{3 \times 3} , {0}_{3 \times 3} ] [ {B}_{i} {A}_{i} {J}_{{P}_{i}}^{- 1} , \begin{matrix}{0}_{3 \times 1} \\ {\hat{z}}_{{Q}_{i}}\end{matrix} ] [ \begin{matrix}{I}_{6 \times 6} & {0}_{6 \times 1} \\ {0}_{1 \times 6} & 1 / {L}_{s}\end{matrix} ] & ( 56 ) \\ {J}_{7 DoF\_r} = [ {0}_{3 \times 3} , {I}_{3 \times 3} ] [ {B}_{i} {A}_{i} {J}_{{P}_{i}}^{- 1} , \begin{matrix}{0}_{3 \times 1} \\ {\hat{z}}_{{Q}_{i}}\end{matrix} ] [ \begin{matrix}{L}_{P} {I}_{6 \times 6} & {0}_{6 \times 1} \\ {0}_{1 \times 6} & 1\end{matrix} ] & ( 57 ) \\ {J}_{8 DoF\_t} = [ {I}_{3 \times 3} , {0}_{3 \times 3} ] {J}_{{h}_{i}} [ \begin{matrix}{I}_{6 \times 6} & {0}_{6 \times 2} \\ {0}_{2 \times 6} & {I}_{2 \times 2} / {L}_{s}\end{matrix} ] & ( 58 ) \\ {J}_{8 DoF\_r} = [ {0}_{3 \times 3} , {I}_{3 \times 3} ] {J}_{{h}_{i}} [ \begin{matrix}{L}_{P} {I}_{6 \times 6} & {0}_{6 \times 2} \\ {0}_{2 \times 6} & {I}_{2 \times 2}\end{matrix} ] & ( 59 )\end{matrix}$$

Organ manipulation with constrained intra-organ motions can be used to evaluate the orbital dexterity when simultaneously using both arms to rotate the eyeball. The evaluation can be designed to address the medical professionals' need to rotate the eye under the microscope in order to obtain a view of peripheral areas of the retina.

The two arms can be predetermined to approach a target point on the retina. The relative position and orientation of the robot end effector with respect to a target point remains constant. The target point on the retina can be selected to be [5π/6,0]^{t}, defined in the eye and attached coordinate system {E}. Frame {E} can be defined similarly as the organ coordinate system {O} and can represent the relative rotation of the eye with respect to {W}. This can cause the target point to rotate together with the eye during a manipulation.

To verify the accuracy of the derivation, a desired rotation velocity of the eye of 10°/sec about the y-axis can be specified and the input joint actuation velocities can be calculated through the inverse of the Jacobian matrix. For rotating the eye by fixing the end effector to a target point two serial robots (e.g., intra-ocular dexterity robots) and the eyeball form a rigid body allowing no relative motion in between. The rates of the serial robot joints can be expected to be zero.

For organ manipulation with unconstrained intra-organ motion, there can be no constraint applied on {dot over (\tilde{x}_{g/t}. Accordingly, it can not be necessary to put limits on the velocities of point gᵢ with respect to a selected target point tᵢ. Further, inserting equation 51 into equation 53 yields:

$$\begin{matrix}M {\dot{q}}_{h} = {N}_{1} {O}_{1} {\dot{q}}_{h} + {N}_{1} {O}_{2} {\dot{x}}_{e} + {N}_{2} {\dot{x}}_{e} \\ where {O}_{1} = [ \begin{matrix}{K}_{1} {J}_{{h}_{1}} & {0}_{5 \times 8} \\ {0}_{5 \times 8} & {K}_{2} {J}_{{h}_{2}}\end{matrix} ] and {O}_{2} = [ \begin{matrix}- {K}_{1} {D}_{1} \\ - {K}_{2} {D}_{2}\end{matrix} ] . & ( 60 ) \\ ( M - {N}_{1} {O}_{1} ) {\dot{q}}_{h} = ( {N}_{1} {O}_{2} + {N}_{2} ) {\dot{x}}_{e} & ( 61 )\end{matrix}$$

For simultaneous organ manipulation and intra-organ operation, both arms can coordinate to manipulate the eyeball. Further, one arm can also operate inside the eye along a specified path. The overall dexterity of the robot utilizing this combined motion can be evaluated. It will be understood that assuming the eye can be rotated about the y-axis by 10°, one arm of the robotic system can scan the retina independently, meaning that there can be a specified relative motion between this arm and the eye. Assuming that the arm inserted through port [π/3,π]^{t} retains fixed in position and orientation with respect to the eye, the arm inserted through port [π/3,π/3]^{t} can coordinate with the previous arm to rotate the eye 10° about the y-axis, but it also scans the retina along the latitude circle θ=5π/6 by 60°. In some embodiments, a single arm can be used to perform an operation.

Transforming the linear and angular velocities from the parallel robot platform center to frame {Qᵢ}, results in:

v_{Q}_{ᵢ}=v_{P}_{ᵢ}+ω_{P}_{ᵢ}×(\vec{pᵢqᵢ}) (62)

ω_{Q}_{ᵢ}=ω_{P}_{ᵢ} (63)

Further, writing equation 62 and equation 63 in matrix form results in the twist of the distal end qᵢ of the connection link:

\dot{x}_{Q}_{ᵢ}=Aᵢ\dot{x}_{P}_{ᵢ} (64)

where Aᵢ=W(\vec{pᵢqᵢ}) can be the twist transformation matrix.

Further, having Bᵢ=W(\vec{qᵢnᵢ}) and Cᵢ=W(\vec{nᵢgᵢ}) the twist of point gᵢ contributed by the parallel robot platform can be calculated. By incorporating the two serial degrees of freedom of the serial robot, the twist of point gᵢ can be obtained:

$${\dot{x}}_{{G}_{i}} = {C}_{i} {B}_{i} {\dot{x}}_{{Q}_{i}} + {C}_{i} [ \begin{matrix}0 \\ {\hat{z}}_{{Q}_{i}}\end{matrix} ] {\dot{q}}_{{s}_{i} 1} + [ \begin{matrix}r {\hat{z}}_{{G}_{i}} \\ {\hat{y}}_{{N}_{i}}\end{matrix} ] {\dot{q}}_{{s}_{i} 2} \qquad ( 65 )$$

Yielding the Jacobian J_{S}_{ᵢ}, of the serial robot as:

\dot{x}_{G}_{ᵢ}=CᵢBᵢ\dot{x}_{Q}_{ᵢ}+J_{S}_{ᵢ}\dot{q}_{S}_{ᵢ} (66)

where

$${J}_{{s}_{i}} = [ \begin{matrix}[ ( - {{n}_{i} {g}_{i}}^{} ) \times ] {\hat{z}}_{{Q}_{i}} & r {\hat{z}}_{{G}_{i}} \\ {\hat{z}}_{{Q}_{i}} & {\hat{y}}_{{N}_{i}}\end{matrix} ]$$

can include the speeds of rotation about the axis of the serial robot tube and the bending of the pre-curved NiTi cannula 520. The hybrid Jacobian matrix relating the twist of point gᵢ and all eight inputs of one arm can be obtained as in equation 34 where Jₕ_{ᵢ}=[CᵢBᵢAᵢJ_{P}_{ᵢ}⁻¹,J_{S}_{ᵢ}] and \dot{q}ₕ_{ᵢ}=[\dot{q}_{P}_{ᵢ}^{t},\dot{q}_{S}_{ᵢ}^{t}]^{t}.

Further, the 5×1 Euler angle parameterization of the desired i^{th} end effector velocity, {dot over (\tilde{x}_{g}_{ᵢ}_{/t}_{ᵢ}, can be related to the general twist of the i^{th} robot end effector, {dot over (\tilde{x}_{g}_{ᵢ}_{/t}_{ᵢ} by the degenerate matrix Kᵢ. The matrix can be derived using a relationship relating the Cartesian angular velocities to the Euler angle velocities:

[ωₓ,ω_{y},ω_{z}]^{t}=Rᵢ[\dot{φ},\dot{θ},\dot{φ}]^{t} (67)

where

$${R}_{i} = [ \begin{matrix}0 & - sin ( {φ}_{i} ) & cos ( {φ}_{i} ) sin ( {θ}_{i} ) \\ 0 & cos ( {φ}_{i} ) & sin ( {φ}_{i} ) sin ( {θ}_{i} ) \\ 1 & 0 & cos ( {θ}_{i} )\end{matrix} ]$$

With the above relationship, the general twist of a system, \dot{X}, can be related to the 6×1 Euler angle twist, [\dot{x}, \dot{y}, ż, \dot{φ}, \dot{θ}, \dot{φ}]^{t}, as follows:

[\dot{x},\dot{y},ż,\dot{φ},\dot{θ},\dot{φ}]^{t}=Sᵢ\dot{x} (68)

where

$${S}_{i} = [ \begin{matrix}I & 0 \\ 0 & {R}_{i}^{- 1}\end{matrix} ] .$$

The 5×1 Euler parameterization used in the aforementioned path planning equation can be derived by applying a 5×6 degenerate matrix to the 6×1 Euler angle twist, as follows:

{dot over (\tilde{x}=[I_{5×5},0_{5×1}][\dot{x},\dot{y},ż,\dot{φ},\dot{θ},\dot{φ}]^{t} (69)

Substituting the relationship between the generalized and the 6×1 Euler angle twist above yields the Matrix Kᵢ as follows:

{dot over (\tilde{x}=Kᵢ\dot{x} (70)

where Kᵢ=[I_{5×5},0_{5×1}]Sᵢ.

As specified above, the constraint that each insertion arm moves at the insertion point only with the velocity equal to the velocity of the organ surface at that point plus any velocity along the insertion needle can be derived as follows. To assist in the development of this constraint, point mᵢ can be defined at the insertion point on the surface of the organ and m′ᵢ can be defined as point on the insertion needle instantaneously coincident with mᵢ. The velocity of m′ᵢ must be equal to the velocity of point mᵢ in the plane perpendicular to the needle axis:

v_{m′}_{ᵢ}_{⊥}=vₘ_{ᵢ}_{⊥} (71)

Taking a dot product in the directions \hat{X}_{Q}_{ᵢ} and ŷ_{Q}_{ᵢ}, yields two independent constraint equations:

\hat{x}_{Q}_{ᵢ}^{t}v_{m′}_{ᵢ}=\hat{x}_{Q}_{ᵢ}^{t}vₘ_{ᵢ} (72)

ŷ_{Q}_{ᵢ}^{t}v_{m′}_{ᵢ}=ŷ_{Q}_{ᵢ}^{t}vₘ_{ᵢ} (73)

These constraints can be expressed in terms of the joint angles and organ velocity by relating the velocities of point mᵢ and m′ᵢ to the robot and organ coordinate systems. The velocity of point m′ᵢ can be related to the velocity of frame {Qᵢ} as

v_{m′}_{ᵢ}=v_{Q}_{ᵢ}+ω_{Q}_{ᵢ}×\vec{qᵢmᵢ} (74)

By substituting the twist of frame {Qᵢ}, equation 74 becomes

v_{m′}_{ᵢ}=[I_{3×3},0_{3×3}]\dot{x}_{Q}_{ᵢ}+Eᵢ[0_{3×3},I_{3×3}]\dot{x}_{Q}_{ᵢ} (75)

where Eᵢ=[(−\vec{qᵢmᵢ}×].

Further, inserting equation 64 and equation 33 and writing in terms of the hybrid joint parameters \dot{q}ₕ_{ᵢ} yields:

v_{m′}_{ᵢ}=Fᵢ\dot{q}ₕ_{ᵢ} (76)

where Fᵢ=([I_{3×3},0_{3×3}]+Eᵢ[0_{3×3},I_{3×3}])AᵢJ_{P}_{ᵢ}⁻¹[I_{6×6},0_{6×2].}

An expression for the velocity of the insertion point m; can be related to the desired organ velocity, yielding:

vₘ_{ᵢ}=\dot{x}ₒₗ+Mᵢ\dot{x}ₒₐ (77)

where Mᵢ=[(−\vec{omᵢ})×].

Further, substituting equation 76 and equation 77 into equation 72 and equation 73 yields the constraint equations given the rigid body motion of the organ-robot system:

\hat{x}_{Q}_{ᵢ}^{t}Fᵢ\dot{q}ₕ_{ᵢ}=\hat{x}_{Q}_{ᵢ}^{t}(\dot{x}ₒₗ+Mᵢ\dot{x}ₒₐ) (78)

ŷ_{Q}_{ᵢ}^{t}Fᵢ\dot{q}ₕ_{ᵢ}=ŷ_{Q}_{ᵢ}^{t}(\dot{x}ₒₗ+Mᵢ\dot{x}ₒₐ) (79)

Vectors \hat{x}_{Q}_{ᵢ} and ŷ_{Q}_{ᵢ} can be put in matrix form as Gᵢ=[\hat{x}_{Q}_{ᵢ},ŷ_{Q}_{_{i]}}^{t}, and matrix Pᵢ can be used to denote Pᵢ=[I_{3×3},Mᵢ].

In some embodiments, stenting can be performed where the size of blood vessels or anatomical features is on the order of 5-900 microns. Some embodiments of the disclosed subject matter can provide, for example, bubble formation, shuts, embolization, clamps, renumerable implants, disposables, and/or drug delivery.

The numbers provided in this paragraph are Current Procedural Terminology (CPT) codes, maintained by the American Medical Association, through the CPT Editorial Panel. These codes are used only as examples. Some embodiments of the disclosed subject matter can be used for, for example, retina surgery, retinal vascular surgery, cannulation, embolization, drug delivery, stenting, angioplasty, bypass surgery, and/or endarterectomy. Some embodiments can be used for, for example, drug delivery device implantation, retinal chip implantation, retinal pigment epithelium cell transplantation, autologous stem cell harvesting (ciliary body), subretinal surgery (instillation of fluid, removal of membranes, translocation), high precision tumor biopsy, therapeutic implantation (i.e. radioactive seed) CPT 678218, robot assisted foreign body removal CPT 65265, robot assisted high precision membrane dissection, such as, for example, retinal detachment repair CPT 67105, 67108, 67112, 67113; proliferative vitreoretinopathy surgery; macular hole repair CPT 67042; epiretinal membrane dissection CPT 67041, and/or robot assisted vitrectomy CPT 67039, 67040; lensectomy CPT 67852. Some embodiments of the disclosed subject matter can be used for, for example, cataract and/or cornea surgery, such as, for example, in automated corneal transplantation {e.g., penetrating keratoplasty, Descemet's stripping endothelial keratoplasty (DSEK), deep lamellar endothelial keratoplasty (DLEK)} CPT 65710, 65730, 65750, 65755; high precision micro-incision phacoemulsification CPT 66984, 66982, 66940, 66850, automated capsulorhexis; and/or iridoplasty CPT 66680, 66682, 66630. Some embodiments can be used for, for example, glaucoma surgery, such as in, for example, micro-seton (tube shunt) placement CPT 66180; micro-filtration surgery CPT 66170, 66172; trabeculotomy/goniotomy CPT 65820; and/or micro-iridotomy or—iridectomy CPT 66625. Some embodiments can be used for, for example, oculoplastics surgery, such as, for example, minimally invasive surgery such as optic nerve sheath fenestration CPT 67038; thyroid decompression surgery CPT 31293; and/or drainage of orbital or sub-periosteal abscess, tumor biopsy. Some embodiments can be used for, for example, robotic assisted oculoplastics surgery, such as, for example, blepharoplasty CPT 15820, 15821; lid laceration repair CPT 66930, 66935, 67930, 67935, 12011-12018, 12051-12057, 13131-13153; orbital fracture repair CPT 21385-21408; brow lift, ptosis repair CPT 67901, 67902; and/or ectropion, entropion, trichiasis repair or biopsy CPT 67961 67966. Some embodiments can, for example, enhance procedures by providing robot assistance. Some embodiments can enable procedures to be performed on humans that may not otherwise have been plausible. Some embodiments can be used for, for example, bypass grafting stem cell harvesting, RPE transplantation, and/or membrane pealing.

Other embodiments, extensions, and modifications of the ideas presented above are comprehended and should be within the reach of one versed in the art upon reviewing the present disclosure. Accordingly, the scope of the disclosed subject matter in its various aspects should not be limited by the examples presented above. The individual aspects of the disclosed subject matter, and the entirety of the disclosed subject matter should be regarded so as to allow for such design modifications and future developments within the scope of the present disclosure. The disclosed subject matter can be limited only by the claims that follow.

What is claimed is:

1. A robot-assisted microsurgical stenting system comprising:
a tele-robotic master and a slave hybrid-robot;
the tele-robotic master comprises at least one user controlled master slave interface;
the slave hybrid-robot comprises at least one robotic arm attached to a frame releasably attachable to a patient; and
the at least one robotic arm comprises a parallel robot and a serial robot, said serial robot comprising a stenting unit.

2. The robot-assisted microsurgical stenting system of claim 1 wherein said stenting unit comprises:
a support tube;
a pre-bent tube positioned within said support tube, said pre-bent tube having an end that that bends when outside said support tube;
a guide wire inserted within said pre-bent tube;
a stent releasably mounted on said guide wire.

3. The robot-assisted microsurgical stenting system of claim 2 further comprising a stent pushing tube positioned around said guide wire for pushing said stent along said guide wire.

4. The robot-assisted microsurgical stenting system of claim 1 wherein the parallel robot comprises a robot having six degrees of freedom and the serial robot comprises a robot having two degrees of freedom.

5. The robot-assisted microsurgical stenting system of claim 2 wherein said pre-bent tube bends in one degree of freedom as it moves outside of said support tube.

6. The robot-assisted microsurgical stenting system of claim 2 wherein at least one of said support tube and said pre-bent tube rotate about their longitudinal axis.

7. The robot-assisted microsurgical stenting system of claim 2 wherein said pre-bent tube bends in one degree of freedom as it moves outside and rotates inside another pre-bent support tube.

8. A robot-assisted microsurgical stenting system comprising:
a tele-robotic master and a slave hybrid-robot;
the tele-robotic master having at least two user controlled master slave interfaces;
the slave hybrid-robot having at least two robotic arms attached to a frame releasably attachable to a patient's head; and
wherein the at least two robotic arms each have a serial robot connected to a parallel robot with at least one of said serial robots comprising a stenting unit.

9. The robot-assisted microsurgical stenting system of claim 8 wherein said stenting unit comprises:
a support tube;
a pre-bent tube positioned within said support tube, said pre-bent tube having an end that that bends when outside said support tube;
a guide wire inserted within said pre-bent tube;
a stent releasably mounted on said guide wire.

10. The robot-assisted microsurgical stenting system of claim 9 further comprising a stent pushing tube positioned around said guide wire for pushing said stent along said guide wire.

11. The robot-assisted microsurgical stenting system of claim 8 wherein the parallel robot comprises a robot having six degrees of freedom and the serial robot comprises a robot having two degrees of freedom.

12. The robot-assisted microsurgical stenting system of claim 9 wherein said pre-bent tube bends in one degree of freedom as it moves outside of said support tube.

13. The robot-assisted microsurgical stenting system of claim 9 wherein at least one of said support tube and said pre-bent tube rotate about their longitudinal axis.

14. The robot-assisted microsurgical stenting system of claim 9 wherein said pre-bent tube bends in one degree of freedom as it moves outside and rotates inside another pre-bent support tube.

15. A method of inserting a stent into a blood vessel comprising the steps of:
inserting a support tube into an organ;
causing a pre-bent tube to extend from said support tube;
causing a guide wire to extend from said pre-bent tube to pierce the blood vessel;
urging a stent mounted around said guide wire to enter the blood vessel;
withdrawing said guide wire from the blood vessel.

16. The method of inserting a stent into a blood vessel of claim 15 wherein said step of urging said stent into a blood vessel comprises causing a stent pushing tube to engage said stent and move said stent into the blood vessel.

17. The method of inserting a stent into a blood vessel of claim 16 wherein said step of urging said stent into a blood vessel comprises rotating said guide wire carrying said stent with a micro-machined screw-like external helix to advance said stent along said guide wire to a desired position.
